(19) United States
(12) Patent Application Publication
(10) Pub. No.: US 2025/0316358 A1
(43) Pub. Date: Oct. 9, 2025

(54) DIABETIC PUMP, APPLICATION, AND PROGRAMMING

(71) Applicant: Diabasal, LLC, Knoxville, TN (US)

(72) Inventors: Richard David Komistek, Knoxville, TN (US); Steven James Michael MacDonald, London (CA); Manh Duc Ta, Knoxville, TN (US)

(73) Assignee: Diabasal, LLC, Knoxville, TN (US)

(21) Appl. No.: 19/098,045

(22) Filed: Apr. 2, 2025

Related U.S. Application Data
(60) Provisional application No. 63/574,636, filed on Apr. 4, 2024.

(51) Int. Cl. G16H 20/17; A61B 5/00; A61B 5/145; A61B 5/1495; A61M 5/142; G16H 10/60; G16H 15/00; G16H 40/63

(52) U.S. Cl. CPC G16H 20/17; A61B 5/14503; A61B 5/1451; A61B 5/14532; A61B 5/1495; A61B 5/6833; A61B 5/7264; A61B 5/7275; A61B 5/7282; A61B 5/742; A61B 5/7475; A61M 5/142; G16H 10/60; G16H 15/00; G16H 40/63; A61B 2560/0214; A61B 2560/0223; A61B 2560/0462; A61M 2005/14208; A61M 2202/0007; A61M 2202/04; A61M 2205/10; A61M 2205/3303; A61M 2205/3327; A61M 2205/502; A61M 2205/583; A61M 2230/201

(57) ABSTRACT

Methods and associated devices for establishing basal rates to be programmed into a diabetic pump that periodically administers insulin to a patient. The instant disclosure also includes diabetic pumps programmed in accordance with the instant disclosure.

### CROSS REFERENCE TO RELATED APPLICATIONS

This application claims the benefit of U.S. Provisional Application No. 63/574,636, filed Apr. 4, 2024, the disclosure of which is incorporated by reference.

### INTRODUCTION TO THE INVENTION

The present disclosure is directed to solutions for problems related to insulin dosing from diabetic pumps, as well as structures and methods to program a diabetic pump to achieve personalized basal delivery inputs for said insulin pumps.

Diabetes is a chronic and potentially life-threatening condition where the body loses its ability to produce insulin, or begins to produce or use insulin less efficiently, resulting in blood glucose levels (BGLs) that are too high (hyperglycemia) to too low (hypoglycemia).

Hypoglycemia means that a person's blood sugar level is low and his/her body (especially his/her brain) is not getting enough oxygen and nutrients. If a person has diabetes, his/her blood sugar can go too low if too much insulin is produced or present. It can also go too low if the person misses a meal and/or is being administered too much insulin. A person can also experience low blood sugar levels if he/she is exercising without administering sugar to offset the sugars consumed via exercising. In addition, certain non-diabetic medications can adversely cause low blood sugar levels.

Symptoms of low blood sugar can start quickly. It may take just 10 to 15 minutes. For persons having diabetes for many years, he/she may not realize that his/her blood sugar is low until it drops very low. If the blood sugar level drops below 70 mg/dL, a person may begin to feel funny, tired, anxious, dizzy, weak, shaky, and/or sweaty even though this blood sugar level is considered the low level of normal. When the blood sugar levels drop to below 60 mg/dL, the foregoing symptoms begin to increase and the person may feel them at increased levels. If one's blood sugar level continues to drop, behavioral changes may be exhibited including, without limitation, increased irritability, difficulty concentrating, and difficulty communicating, and difficulty maintaining one's balance.

If one's blood sugar level drops too low, around 30-40 mg/dL, a person could pass out. A very low blood sugar level can be a scary time for a diabetic because he/she may be cognitively impaired and unable to realize what is happening and how to resolve the low blood sugar level. As the blood sugar levels continue to drop, a seizure or stroke could occur.

Another concern is if a person experiences a low blood sugar level during the night, and they may wake up tired or with a headache. They may also sweat so much during the night that their pajamas or sheets are damp when you wake up. Unfortunately, as a diabetic gets older the sign of low glucose occurring because of sweating does dissipate often and may no longer occur.

Low glucose can be treated by eating or drinking something that has carbohydrates, but it is best if it contains high amounts of sugar concentrate. Therefore, these should be quick-sugar foods. One of the best ways to raise blood glucose is drinking orange juice. A person should continue to monitor their blood sugar level until their level has returned to normal.

Over time, BGLs above the normal range can damage your eyes, kidneys and nerves, and can also cause heart disease and stroke. Every 17 seconds, another individual is diagnosed with diabetes. Each day approximately 5,082 people are diagnosed with diabetes. About 1.9 million people will be diagnosed this year. Diabetes is the fastest-growing chronic condition in the world. The main types of diabetes are type 1, type 2, and gestational diabetes.

Approximately 537 million adults (20-79 years) are living with diabetes. The total number of people living with diabetes is projected to rise to 643 million by 2030 and 783 million by 2045.

Type 1 diabetes develops when the cells of the pancreas stop producing insulin. Without insulin, glucose cannot enter the cells of the muscles for energy. Instead, glucose levels rise in the blood causing a person to become extremely unwell. Type 1 diabetes is life-threatening if insulin is not replaced. People with type 1 diabetes need to inject insulin for the rest of their lives.

Type 1 diabetes often occurs in children and people under 30 years of age, but it can occur at any age. This condition is not caused by lifestyle factors. Its exact cause is not known, but research shows that something in the environment can trigger it in a person that has a genetic risk.

The body's immune system attacks and destroys the beta cells of the pancreas after the person gets a virus because it sees the cells as foreign. Most people diagnosed with type 1 diabetes do not have family members with this condition. In 2021, there were about 8.4 million individuals worldwide with type 1 diabetes: of these 1.5 million (18%) were younger than 20 years, 5.4 million (64%) were aged 20-59 years, and 1.6 million (19%) were aged 60 years or older. This number is predicted to increase to 13.5-17.4 million people living with Type 1 diabetes by 2040.

Type 2 diabetes develops when the pancreas does not make enough insulin and the insulin that is made does not work as well as it should (also known as insulin resistance). As a result, the glucose begins to rise above normal levels in the blood. Half the people with type 2 diabetes do not know they have the condition because they have no symptoms.

Type 2 diabetes (once known as adult-onset diabetes) affects 85 to 90% of all people with diabetes. People who develop type 2 diabetes are very likely to also have someone in their family with the condition. It is considered a lifestyle condition because being overweight and not exercising enough increases the risk of developing type 2 diabetes. People from certain ethnic backgrounds, such as Aboriginal or Torres Strait Islander, Polynesian, Asian or Indian are more likely to develop type 2 diabetes.

When first diagnosed, many people with type 2 diabetes can manage their condition with a healthy diet and increased physical activity. Over time, however, most people with type 2 diabetes will need diabetes tablets to help keep their BGLs in the target range. (Regular blood glucose monitoring may be necessary in order to keep track of the effectiveness of the treatment.) The starting time for diabetes tablets varies according to individual need. About 50% of people with type 2 diabetes need insulin injections within 6 to 10 years of diagnosis.

Gestational diabetes occurs in about 5 to 10% of pregnant women, and usually goes away after the birth of the baby. Women who have had gestational diabetes have an increased risk of developing type 2 diabetes later on.

The management of gestational diabetes includes seeing a dietitian to assist with healthy eating strategies to help manage BGLs. Where possible, regular exercise such as walking also helps. Measuring BGLs with a blood glucose meter gives information about whether these management strategies are able to keep BGLs in the recommended range. Some women may need to also inject insulin to help manage their BGLs until their baby is born.

Insulin is a hormone used to manage type 1 and, in some cases, type 2 diabetes. There are several important factors related to insulin. Insulin is a hormone that lowers glucose in your blood and can be injected or inhaled, replacing what the body makes naturally. People with type 1 diabetes must take insulin to survive. About half the people with type 2 diabetes will need to take insulin at some point in their lives. Taking insulin doesn't mean a person has done a bad job managing their diabetes, but rather, your body has gotten to a point with the disease where it needs extra help. Insulin is safe and one of the most effective ways to lower blood glucose. It is measured in units just as milk is measured in pints and quarts. Insulin is made in different strengths. Most people use a strength called U-100. Insulins come in several different types. Some are faster-working and last for a shorter period of time, while others are slower-working and last for a longer period of time. Different companies make different types of insulin. Diabetics are advised to always use the same brand and type of insulin that your provider has prescribed. Different injection sites (leg, stomach, etc.) may absorb some types of insulin at faster or slower rates, but normally the closer the injection is to the heart, the quicker it will absorb in the body. The main side effect of insulin is that it can cause low BGLs. Knowing how to recognize and treat low glucose levels is an important part of taking insulin.

Rapid acting insulin is a type of insulin that starts to work within 15 minutes of injection and peaks between 1 to 3 hours after injection, but this does vary from subject to subject. The duration can be anywhere from 3 to 7 hours. Some resources further divide rapid acting insulin into very rapid acting with an onset between 15 to 20 minutes from injection and rapid acting with an onset of action between 15 to 30 minutes. It is important for a diabetic to understand the absorption rate for them personally and to understand that there is a delay before the body absorbs the insulin at its peak. Examples include insulin lispro, (brand names: Admelog, Humalog), lispro-aabc (brand name: Lyumjev), insulin aspart (brand names: Fiasp, NovoLog), and insulin glulisine (brand name: Apidra). In this list, Fiasp and Lyumjev are considered very rapid-acting insulins.

A very rapid-acting inhaled insulin is also available. It starts to work within 10 to 15 minutes, has a peak within 35 to 45 minutes, and its duration is between 1.5 to 3 hours. This rapid acting inhaled insulin, known by the brand name Afrezza, is an inhaled powder form of regular human insulin.

Short acting insulin is a type of insulin that takes about 30 minutes to start working and peaks at about 2 to 3 hours after injection, but can peak as early as one hour. The effective duration is approximately 5 to 8 hours, but can, in certain circumstances, last only 4 hours. Examples include regular insulin (brand names: Humulin R, Novolin R).

Intermediate acting insulin is a type of insulin that takes about 2 to 4 hours to start working and peaks at about 4 to 12 hours after injection. The effective duration is 12 to 18 hours. Examples include NPH insulin (brand names: Humulin N, Novolin N).

Long acting insulin is a type of insulin that starts working several hours after injection and can last up to 24 hours or more. Examples include insulin glargine (brand name: Lantus), insulin detemir (brand names: Levemir), and insulin degludec (brand name: Tresiba). Longer duration, long-acting insulins are on the horizon, including a weekly long-acting insulin.

There are also combination types of insulin combining different types of insulin into one injection. This type of insulin typically starts working within 5 to 60 minutes. The peaks vary and the duration is anywhere from 10 to 24 hours. Examples include the brand names: Humalog Mix 75/25, Humalog Mix 50/50, NovoLog Mix 70/30, and Novolin 70/30.

People with diabetes must also give bolus and basal doses and it is important for them to understand the differences, how their body best absorbs these types of doses, and how to administer them.

A bolus is a single, large dose of medicine. For a person with diabetes, a bolus is a dose of insulin taken to handle a rise in blood glucose (a type of sugar), like the one that happens during eating. A bolus is given as a shot or through an insulin pump.

Basal doses for diabetes can be referred to as background insulin. Your pancreas normally makes set amounts of insulin around the clock. Basal insulin mimics that process, and your body absorbs it slowly and uses it throughout the day.

For diabetic patients not using an insulin pump, using a needle, they administer long-term insulin that will be slowly released and absorbed by the body, throughout the day for 24 hours, as their basal insulin. Therefore, their body is absorbing this insulin throughout the day. Then, during meals or to correct high blood sugar episodes, using a needle, they will administer a bolus insulin dose that is a fast-acting insulin that is fully absorbed by the body in 3-4 hours. Therefore, the body receives both basal and bolus insulin doses throughout the day.

Referring to FIG. 1, when using an exemplary insulin pump 100, a controller 102 of the pump includes a display screen 104 providing a graphical user interface (GUI) with many data input options for a user, two of which include Bolus 106 and Basal 108. When choosing Bolus 106, the display screen 104 GUI allows the user to administer insulin in an increment previously programmed by the user or a default amount pre-programmed by the pump manufacturer. By way of example, a common increment for Bolus is 0.1 units of insulin (or 0.00347 milligrams of insulin).

Referencing FIGS. 1 and 2, unlike Bolus 106, the Basal 108 dose is pre-programmed as part of the pump controller 102. Diabetic patients using an insulin pump 100 may program basal rates for a 24-hour period (see FIG. 2). By way of example, a pump user may program the same or a different rate for each hour of a 24-hour period and the pump controller 102 will utilize this one-hour rate divided by twelve to dose insulin repetitively to the user every five minutes for that hour. Then, for each subsequent hour, the pump controller 102 will use the programmed rate for that hour, divided by twelve, to dose insulin to the user in five-minute increments. This process is repeated and restarts at the end of a 24-hour period. In cases where the user programs the pump controller 102 with rates that vary across the twenty-four hour duration, the user can program twenty-four or fewer rates. In a case where the programming increments are thirty minutes, the user would be able to program forty-eight or fewer rates—each corresponding to a thirty minute interval. For example, if the pump controller 102 is programmed to administer 1.2 units of insulin between 1:00 pm to 2:00 pm (one hour), the controller will direct the pump to administer 0.1 units (1.2/12) of insulin every five minutes. Similarly, if the controller 102 is programmed for 1.8 units of insulin from 2:00 pm to 3:00 pm (one hour), then the controller will direct the pump to administer 0.15 units (1.8/12) of insulin every five minutes. FIG. 2 depicts a first exemplary programming increment 120 where the user programs the pump to deliver 1.1 units of insulin each hour between 12 AM and 2 AM, while this same programming increment will deliver 1.2 units of insulin each hour between 2 AM and 6 AM. Similarly, a second programming increment 130 where the user programs the pump to deliver 1.6 units of insulin each hour between 10 AM and 12 PM, while this same programming increment will deliver 1.7 units of insulin each hour between 12 PM and 1 PM.

Although these basal rates are pre-programmed or entered by a pump user, these rates are essentially guesses as to how much insulin should administered per hour. But each person absorbs insulin at different rates due to body type, shape, diet, exercise, etc. Also, if the basal pump rates are programmed for normal daily activity by the user, the glucose values will necessarily change as the person ages, as the person exercises, if the person is not feeling well, etc. Therefore, the inventors of the instant disclosure and inventions hypothesize that the basal rates should be programmed using historical person-specific blood sugar data and vary based upon the time of day, meal times, and activities or health of the diabetic. Also, basal rates and basal patterns are different for each person and should be personalized based on absorption rate, activity level, their job (sitting all day vs. being active), travel (requiring sitting and being inactive and if flying, changes due to altitude), eating patterns, among other variables.

Although patients are taught how to use a pump and are trained with respect to utilization of various aspects such as programming insulin levels, determining the status of the pump, the amount of insulin still available in the reservoir, pump notifications, how to suspend delivery, reviewing history of insulin administered, replacing a reservoir and other components, delivery settings and other settings such as time and date, no one teaches a person with diabetes how to determine how much insulin they need to program for basal rates throughout the day. The person with diabetes actually guesses as to how much insulin they should program each hour for a basal rate. More studious users of a pump may modify these rates, but again, not based on scientific data but rather their opinion of what is occurring throughout the day with regard to glucose levels.

More recently, insulin pumps can take in feedback using continuous glucose monitoring (CGM) sensors that determine glucose levels every 1-5 minutes. Then, based on this reading, the pump will administer small amounts of insulin that are viewed as basal doses. There are two main concerns for this option where the insulin pump determines the amount of insulin needed and administers this amount:
1. If the sensor reading is incorrect, the patient will get the wrong amount of insulin.
2. This method is reactive and not proactive as the sensor gives the pump a glucose value and the pump then determines, essentially, what the corrective basal dose should be, but insulin absorption has a delay. Therefore, if a person with diabetes administers 1 unit of insulin, it will take the body at least four hours to absorb this full amount of insulin. This is of course if the pump has fast-acting insulin in the reservoir. By using this process, the correction pertaining to the full absorption is always four hours behind.

Even if a person using a control feedback insulin pump/CGM sensor combination, the patient still is required to program basal rates into their insulin pump. It is not safe to not utilize proper basal rates and allow the CGM to provide feedback to the pump in a closed loop to administer insulin into the body. If the CGM reading is incorrect, the patient will get the wrong amount of insulin. Therefore, if the pump gives a patient too much insulin, they could experience a low blood glucose level that could lead to a patient experiencing hypoglycemia.

It is a first aspect of the present invention to provide a method of creating an insulin dosing profile using at least one of a computer and a portable electronic device, the method comprising: (i) processing historical blood glucose level data output from a continuous glucose monitoring device over a first period of time for a first person to segment the historical blood glucose level data into basal controlled glucose patterns and bolus controlled glucose patterns; (ii) using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump; and, (iii) optionally, programming a controller of the insulin dosing pump in accordance with the suggested insulin dosing regimen.

In a more detailed embodiment of the first aspect, the first period of time includes at least seven consecutive days and preferably includes at least twenty consecutive days. In yet another more detailed embodiment, processing historical blood glucose level data output from the continuous glucose monitoring device over the first period of time for the first person includes identifying missing blood glucose level data, the method further comprises interpolating, if necessary, to create blood glucose level data substituted for the missing blood glucose level data, thus creating a complete set of historical blood glucose level data for the first period of time, where segmenting the historical blood glucose level data includes segmenting the complete set of historical blood glucose level data into basal controlled glucose patterns and bolus controlled glucose patterns. In a further detailed embodiment, identifying the missing blood glucose level data includes identifying whether the missing blood glucose level data is attributable to either a random error or a systematic error. In still a further detailed embodiment, the missing blood glucose level data is attributable to a systematic error, initiating a continuous glucose monitoring device calibration instruction. In a more detailed embodiment, the missing blood glucose level data is attributable to a systematic error, initiating message to a user indicating the continuous glucose monitoring device is not properly working. In a more detailed embodiment, the method further includes processing the historical blood glucose level data output from the continuous glucose monitoring device includes transforming raw data from the continuous glucose monitoring device into a structured dataset comprising the historical blood glucose level data. In another more detailed embodiment, the historical blood glucose level data is comprised of data in a tabular form that includes glucose measurements and corresponding time stamps. In yet another more detailed embodiment, interpolating, if necessary, to create blood glucose level data substituted for the missing blood glucose level data includes using linear interpolation to create the blood glucose level data where the missing data is attributable to a random occurrence. In still another more detailed embodiment, the method further includes applying a data smoothing operation to the complete set of historical blood glucose level data to mitigate large fluctuations in blood glucose level data at adjacent times.

In yet another more detailed embodiment of the first aspect, applying the data smoothing operation includes excluding blood glucose level data above a predetermined threshold. In yet another more detailed embodiment, segmenting the historical blood glucose level data into basal controlled glucose patterns and bolus controlled glucose patterns includes identifying meal events using time stamps associated with the historical blood glucose level data. In a further detailed embodiment, identifying meal events includes using time of day and rate of change of historical blood glucose level data. In still a further detailed embodiment, the bolus controlled glucose patterns comprises historical blood glucose level data attributable to meal events. In a more detailed embodiment, segmenting the historical blood glucose level data into basal controlled glucose patterns and bolus controlled glucose patterns includes identifying a rate of change in the basal controlled glucose patterns. In a more detailed embodiment, identifying the rate of change in the basal controlled glucose patterns includes segmenting the basal controlled glucose patterns into accelerated glucose level rate changes and decelerated glucose level rate change. In another more detailed embodiment, the method further includes calculating a mean deviation for the historical blood glucose level data within the basal controlled glucose patterns. In yet another more detailed embodiment, the method further includes calculating a standard variation for the historical blood glucose level data within the basal controlled glucose patterns. In still another more detailed embodiment, at least one of the mean deviation and the standard deviation is utilized to establish the suggested insulin dosing regimen for the insulin dosing pump.

In a more detailed embodiment of the first aspect, segmenting the historical blood glucose level data into basal controlled glucose patterns and bolus controlled glucose patterns includes identifying a duration that historical blood glucose level data remained above a predetermined threshold, where values at or above the predetermined threshold results in the first person being hyperglycemic. In yet another more detailed embodiment, segmenting the historical blood glucose level data into basal controlled glucose patterns and bolus controlled glucose patterns includes identifying a duration that historical blood glucose level data remained below a predetermined threshold, where values at or below the predetermined threshold results in the first person being hypoglycemic. In a further detailed embodiment, establishing the suggested insulin dosing regimen for the insulin dosing pump includes using an input regarding a type of insulin dosed to the first person. In still a further detailed embodiment, the type of insulin dosed to the first person is at least one of very rapid-acting insulin, short acting insulin, intermediate acting insulin, and long acting insulin. In a more detailed embodiment, establishing the suggested insulin dosing regimen for the insulin dosing pump includes using an input regarding an absorption rate of insulin dosed to the first person. In a more detailed embodiment, the input regarding the absorption rate of insulin is derived from the historical blood glucose level data and the historical insulin dosing data. In another more detailed embodiment, establishing the suggested insulin dosing regimen for the insulin dosing pump includes using an input regarding a physical condition of the first person.

In a more detailed embodiment of the first aspect, the physical condition of the first person includes an input regarding whether the first person was ill during the first period of time. In yet another more detailed embodiment, establishing the suggested insulin dosing regimen for the insulin dosing pump includes establishing a suggested insulin dosing regimen when ill for the first person. In a further detailed embodiment, the physical condition of the first person includes an input regarding whether the first person exercised during the first period of time. In still a further detailed embodiment, establishing the suggested insulin dosing regimen for the insulin dosing pump includes establishing a suggested insulin dosing regimen when exercising for the first person. In a more detailed embodiment, establishing the suggested insulin dosing regimen for the insulin dosing pump includes using an input regarding a type of insulin pump the first person uses. In a more detailed embodiment, the method further includes inputting the historical blood glucose level data output from the continuous glucose monitoring device over the first period of time for the first person, and inputting the historical insulin dosing for the first person over the first period of time. In another more detailed embodiment, the method further includes processing additional historical blood glucose level data output from the continuous glucose monitoring device over a second period of time for the first person to segment the additional historical blood glucose level data into additional basal controlled glucose patterns and additional bolus controlled glucose patterns, and using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump, and optionally, reprogramming the insulin dosing pump in accordance with the suggested revised insulin dosing regimen. In yet another more detailed embodiment, the method further includes generating a report that includes graphical feedback showing how the additional historical blood glucose level data changes with time. In still another more detailed embodiment, the report includes at least one of a mean deviation and a standard deviation of blood glucose rates of change using the additional historical blood glucose level data.

It is a second aspect of the present invention to provide a method for executing a computer application, the method comprising: (i) running a computer application on a portable electronic device comprising executing a software application embodied on the portable electronic device which causes the portable electronic device to perform the steps of: (a) processing historical blood glucose level data output from a continuous glucose monitoring device over a first period of time for a first person to segment the historical blood glucose level data into basal controlled glucose patterns and bolus controlled glucose patterns; (b) using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump; and, (c) optionally, programming a controller of the insulin dosing pump in accordance with the suggested insulin dosing regimen.

In a more detailed embodiment of the second aspect, the first period of time includes at least seven consecutive days and preferably includes at least twenty consecutive days. In yet another more detailed embodiment, processing historical blood glucose level data output from the continuous glucose monitoring device over the first period of time for the first person includes identifying missing blood glucose level data, the method further comprises interpolating, if necessary, to create blood glucose level data substituted for the missing blood glucose level data, thus creating a complete set of historical blood glucose level data for the first period of time, where segmenting the historical blood glucose level data includes segmenting the complete set of historical blood glucose level data into basal controlled glucose patterns and bolus controlled glucose patterns. In a further detailed embodiment, identifying the missing blood glucose level data includes identifying whether the missing blood glucose level data is attributable to either a random error or a systematic error. In still a further detailed embodiment, the missing blood glucose level data is attributable to a systematic error, initiating a continuous glucose monitoring device calibration instruction. In a more detailed embodiment, the missing blood glucose level data is attributable to a systematic error, initiating message to a user indicating the continuous glucose monitoring device is not properly working. In a more detailed embodiment, the method further includes processing the historical blood glucose level data output from the continuous glucose monitoring device includes transforming raw data from the continuous glucose monitoring device into a structured dataset comprising the historical blood glucose level data. In another more detailed embodiment, the historical blood glucose level data is comprised of data in a tabular form that includes glucose measurements and corresponding time stamps. In yet another more detailed embodiment, interpolating, if necessary, to create blood glucose level data substituted for the missing blood glucose level data includes using linear interpolation to create the blood glucose level data where the missing data is attributable to a random occurrence. In still another more detailed embodiment, the method further includes applying a data smoothing operation to the complete set of historical blood glucose level data to mitigate large fluctuations in blood glucose level data at adjacent times.

In yet another more detailed embodiment of the second aspect, applying the data smoothing operation includes excluding blood glucose level data above a predetermined threshold. In yet another more detailed embodiment, segmenting the historical blood glucose level data into basal controlled glucose patterns and bolus controlled glucose patterns includes identifying meal events using time stamps associated with the historical blood glucose level data. In a further detailed embodiment, identifying meal events includes using time of day and rate of change of historical blood glucose level data. In still a further detailed embodiment, the bolus controlled glucose patterns comprises historical blood glucose level data attributable to meal events. In a more detailed embodiment, segmenting the historical blood glucose level data into basal controlled glucose patterns and bolus controlled glucose patterns includes identifying a rate of change in the basal controlled glucose patterns. In a more detailed embodiment, identifying the rate of change in the basal controlled glucose patterns includes segmenting the basal controlled glucose patterns into accelerated glucose level rate changes and decelerated glucose level rate change. In another more detailed embodiment, the method further includes calculating a mean deviation for the historical blood glucose level data within the basal controlled glucose patterns. In yet another more detailed embodiment, the method further includes calculating a standard variation for the historical blood glucose level data within the basal controlled glucose patterns. In still another more detailed embodiment, at least one of the mean deviation and the standard deviation is utilized to establish the suggested insulin dosing regimen for the insulin dosing pump.

In a more detailed embodiment of the second aspect, segmenting the historical blood glucose level data into basal controlled glucose patterns and bolus controlled glucose patterns includes identifying a duration that historical blood glucose level data remained above a predetermined threshold, where values at or above the predetermined threshold results in the first person being hyperglycemic. In yet another more detailed embodiment, segmenting the historical blood glucose level data into basal controlled glucose patterns and bolus controlled glucose patterns includes identifying a duration that historical blood glucose level data remained below a predetermined threshold, where values at or below the predetermined threshold results in the first person being hypoglycemic. In a further detailed embodiment, establishing the suggested insulin dosing regimen for the insulin dosing pump includes using an input regarding a type of insulin dosed to the first person. In still a further detailed embodiment, the type of insulin dosed to the first person is at least one of very rapid-acting insulin, short acting insulin, intermediate acting insulin, and long acting insulin. In a more detailed embodiment, establishing the suggested insulin dosing regimen for the insulin dosing pump includes using an input regarding an absorption rate of insulin dosed to the first person. In a more detailed embodiment, the input regarding the absorption rate of insulin is derived from the historical blood glucose level data and the historical insulin dosing data. In another more detailed embodiment, establishing the suggested insulin dosing regimen for the insulin dosing pump includes using an input regarding a physical condition of the first person.

In a more detailed embodiment of the second aspect, the physical condition of the first person includes an input regarding whether the first person was ill during the first period of time. In yet another more detailed embodiment, establishing the suggested insulin dosing regimen for the insulin dosing pump includes establishing a suggested insulin dosing regimen when ill for the first person. In a further detailed embodiment, the physical condition of the first person includes an input regarding whether the first person exercised during the first period of time. In still a further detailed embodiment, establishing the suggested insulin dosing regimen for the insulin dosing pump includes establishing a suggested insulin dosing regimen when exercising for the first person. In a more detailed embodiment, establishing the suggested insulin dosing regimen for the insulin dosing pump includes using an input regarding a type of insulin pump the first person uses. In a more detailed embodiment, the method further includes inputting the historical blood glucose level data output from the continuous glucose monitoring device over the first period of time for the first person, and inputting the historical insulin dosing for the first person over the first period of time. In another more detailed embodiment, the method further includes processing additional historical blood glucose level data output from the continuous glucose monitoring device over a second period of time for the first person to segment the additional historical blood glucose level data into additional basal controlled glucose patterns and additional bolus controlled glucose patterns, and using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump, and optionally, reprogramming the insulin dosing pump in accordance with the suggested revised insulin dosing regimen. In yet another more detailed embodiment, the method further includes generating a report that includes graphical feedback showing how the additional historical blood glucose level data changes with time. In still another more detailed embodiment, the report includes at least one of a mean deviation and a standard deviation of blood glucose rates of change using the additional historical blood glucose level data.

It is a third aspect of the present invention to provide a computer program product for establishing a glucose dosing regimen for an insulin pump, the computer program product comprising: (i) a non-transitory computer readable medium encoded with computer executable code, the code configured to enable the execution of: (a) processing historical blood glucose level data output from a continuous glucose monitoring device over a first period of time for a first person to segment the historical blood glucose level data into basal controlled glucose patterns and bolus controlled glucose patterns; and, (b) using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump.

In a more detailed embodiment of the third aspect, the first period of time includes at least seven consecutive days and preferably includes at least twenty consecutive days. In yet another more detailed embodiment, processing historical blood glucose level data output from the continuous glucose monitoring device over the first period of time for the first person includes identifying missing blood glucose level data, the code is configured to enable the execution of the act of interpolating, if necessary, to create blood glucose level data substituted for the missing blood glucose level data, thus creating a complete set of historical blood glucose level data for the first period of time, where segmenting the historical blood glucose level data includes segmenting the complete set of historical blood glucose level data into basal controlled glucose patterns and bolus controlled glucose patterns. In a further detailed embodiment, identifying the missing blood glucose level data includes identifying whether the missing blood glucose level data is attributable to either a random error or a systematic error. In still a further detailed embodiment, the missing blood glucose level data is attributable to a systematic error, initiating a continuous glucose monitoring device calibration instruction. In a more detailed embodiment, the missing blood glucose level data is attributable to a systematic error, initiating message to a user indicating the continuous glucose monitoring device is not properly working. In a more detailed embodiment, the code is configured to enable the execution of the act of processing the historical blood glucose level data output from the continuous glucose monitoring device includes transforming raw data from the continuous glucose monitoring device into a structured dataset comprising the historical blood glucose level data. In another more detailed embodiment, the historical blood glucose level data is comprised of data in a tabular form that includes glucose measurements and corresponding time stamps. In yet another more detailed embodiment, interpolating, if necessary, to create blood glucose level data substituted for the missing blood glucose level data includes using linear interpolation to create the blood glucose level data where the missing data is attributable to a random occurrence. In still another more detailed embodiment, the code is configured to enable the execution of the act of applying a data smoothing operation to the complete set of historical blood glucose level data to mitigate large fluctuations in blood glucose level data at adjacent times.

In yet another more detailed embodiment of the third aspect, applying the data smoothing operation includes excluding blood glucose level data above a predetermined threshold. In yet another more detailed embodiment, segmenting the historical blood glucose level data into basal controlled glucose patterns and bolus controlled glucose patterns includes identifying meal events using time stamps associated with the historical blood glucose level data. In a further detailed embodiment, identifying meal events includes using time of day and rate of change of historical blood glucose level data. In still a further detailed embodiment, the bolus controlled glucose patterns comprises historical blood glucose level data attributable to meal events. In a more detailed embodiment, segmenting the historical blood glucose level data into basal controlled glucose patterns and bolus controlled glucose patterns includes identifying a rate of change in the basal controlled glucose patterns. In a more detailed embodiment, identifying the rate of change in the basal controlled glucose patterns includes segmenting the basal controlled glucose patterns into accelerated glucose level rate changes and decelerated glucose level rate change. In another more detailed embodiment, the code is configured to enable the execution of the act of calculating a mean deviation for the historical blood glucose level data within the basal controlled glucose patterns. In yet another more detailed embodiment, the code is configured to enable the execution of the act of calculating a standard variation for the historical blood glucose level data within the basal controlled glucose patterns. In still another more detailed embodiment, at least one of the mean deviation and the standard deviation is utilized to establish the suggested insulin dosing regimen for the insulin dosing pump.

In a more detailed embodiment of the third aspect, segmenting the historical blood glucose level data into basal controlled glucose patterns and bolus controlled glucose patterns includes identifying a duration that historical blood glucose level data remained above a predetermined threshold, where values at or above the predetermined threshold results in the first person being hyperglycemic. In yet another more detailed embodiment, segmenting the historical blood glucose level data into basal controlled glucose patterns and bolus controlled glucose patterns includes identifying a duration that historical blood glucose level data remained below a predetermined threshold, where values at or below the predetermined threshold results in the first person being hypoglycemic. In a further detailed embodiment, establishing the suggested insulin dosing regimen for the insulin dosing pump includes using an input regarding a type of insulin dosed to the first person. In still a further detailed embodiment, the type of insulin dosed to the first person is at least one of very rapid-acting insulin, short acting insulin, intermediate acting insulin, and long acting insulin. In a more detailed embodiment, establishing the suggested insulin dosing regimen for the insulin dosing pump includes using an input regarding an absorption rate of insulin dosed to the first person. In a more detailed embodiment, the input regarding the absorption rate of insulin is derived from the historical blood glucose level data and the historical insulin dosing data. In another more detailed embodiment, establishing the suggested insulin dosing regimen for the insulin dosing pump includes using an input regarding a physical condition of the first person.

In a more detailed embodiment of the third aspect, the physical condition of the first person includes an input regarding whether the first person was ill during the first period of time. In yet another more detailed embodiment, establishing the suggested insulin dosing regimen for the insulin dosing pump includes establishing a suggested insulin dosing regimen when ill for the first person. In a further detailed embodiment, the physical condition of the first person includes an input regarding whether the first person exercised during the first period of time. In still a further detailed embodiment, establishing the suggested insulin dosing regimen for the insulin dosing pump includes establishing a suggested insulin dosing regimen when exercising for the first person. In a more detailed embodiment, establishing the suggested insulin dosing regimen for the insulin dosing pump includes using an input regarding a type of insulin pump the first person uses. In a more detailed embodiment, the code is configured to enable the execution of the acts of inputting the historical blood glucose level data output from the continuous glucose monitoring device over the first period of time for the first person, and inputting the historical insulin dosing for the first person over the first period of time. In another more detailed embodiment, the code is configured to enable the execution of the acts of processing additional historical blood glucose level data output from the continuous glucose monitoring device over a second period of time for the first person to segment the additional historical blood glucose level data into additional basal controlled glucose patterns and additional bolus controlled glucose patterns, and using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump, and optionally, reprogramming the insulin dosing pump in accordance with the suggested revised insulin dosing regimen. In yet another more detailed embodiment, the code is configured to enable the execution of the act of generating a report that includes graphical feedback showing how the additional historical blood glucose level data changes with time. In still another more detailed embodiment, the report includes at least one of a mean deviation and a standard deviation of blood glucose rates of change using the additional historical blood glucose level data.

In a more detailed embodiment of the third aspect, the code is configured to enable the execution of the act of generating programming code configured to be executable by a controller of the insulin dosing pump to dose insulin consistent with the suggested insulin dosing regimen. In yet another more detailed embodiment, the code is configured to enable the execution of the acts of processing additional historical blood glucose level data output from the continuous glucose monitoring device over a second period of time for the first person to segment the additional historical blood glucose level data into additional basal controlled glucose patterns and additional bolus controlled glucose patterns, and using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump, and generating additional programming code configured to be executable by the controller of the insulin dosing pump to dose insulin consistent with the suggested revised insulin dosing regimen.

It is a fourth aspect of the present invention to provide an insulin pump with a controller programmed with executable code configured to enable execution of the following acts: (i) processing historical blood glucose level data output from a continuous glucose monitoring device over a first period of time for a first person to segment the historical blood glucose level data into basal controlled glucose patterns and bolus controlled glucose patterns; (ii) using the historical blood glucose level data within the basal controlled glucose patterns and historical insulin dosing data over the first period of time, but excluding using the historical blood glucose level data within the bolus controlled glucose patterns, to establish an insulin dosing regimen; and, (iii) dosing insulin consistent with the insulin dosing regimen.

In a more detailed embodiment of the fourth aspect, the first period of time includes at least seven consecutive days and preferably includes at least twenty consecutive days. In yet another more detailed embodiment, processing historical blood glucose level data output from the continuous glucose monitoring device over the first period of time for the first person includes identifying missing blood glucose level data, the controller is programmed with executable code configured to enable execution of a further act comprising: interpolating, if necessary, to create blood glucose level data substituted for the missing blood glucose level data, thus creating a complete set of historical blood glucose level data for the first period of time, and segmenting the historical blood glucose level data includes segmenting the complete set of historical blood glucose level data into basal controlled glucose patterns and bolus controlled glucose patterns. In a further detailed embodiment, identifying the missing blood glucose level data includes identifying whether the missing blood glucose level data is attributable to either a random error or a systematic error. In still a further detailed embodiment, the missing blood glucose level data is attributable to a systematic error, initiating a continuous glucose monitoring device calibration instruction. In a more detailed embodiment, the missing blood glucose level data is attributable to a systematic error, initiating message to a user of the insulin pump indicating the continuous glucose monitoring device is not properly working. In a more detailed embodiment, the controller is programmed with executable code configured to enable execution of a further act comprising processing the historical blood glucose level data output from the continuous glucose monitoring device includes transforming raw data from the continuous glucose monitoring device into a structured dataset comprising the historical blood glucose level data. In another more detailed embodiment, the historical blood glucose level data is comprised of data in a tabular form that includes glucose measurements and corresponding time stamps. In yet another more detailed embodiment, interpolating, if necessary, to create blood glucose level data substituted for the missing blood glucose level data includes using linear interpolation to create the blood glucose level data where the missing data is attributable to a random occurrence. In still another more detailed embodiment, the controller is programmed with executable code configured to enable execution of a further act comprising applying a data smoothing operation to the complete set of historical blood glucose level data to mitigate large fluctuations in blood glucose level data at adjacent times.

In yet another more detailed embodiment of the fourth aspect, applying the data smoothing operation includes excluding blood glucose level data above a predetermined threshold. In yet another more detailed embodiment, segmenting the historical blood glucose level data into basal controlled glucose patterns and bolus controlled glucose patterns includes identifying meal events using time stamps associated with the historical blood glucose level data. In a further detailed embodiment, identifying meal events includes using time of day and rate of change of historical blood glucose level data. In still a further detailed embodiment, the bolus controlled glucose patterns comprises historical blood glucose level data attributable to meal events. In a more detailed embodiment, segmenting the historical blood glucose level data into basal controlled glucose patterns and bolus controlled glucose patterns includes identifying a rate of change in the basal controlled glucose patterns. In a more detailed embodiment, identifying the rate of change in the basal controlled glucose patterns includes segmenting the basal controlled glucose patterns into accelerated glucose level rate changes and decelerated glucose level rate change. In another more detailed embodiment, the controller is programmed with executable code configured to enable execution of a further act comprising calculating a mean deviation for the historical blood glucose level data within the basal controlled glucose patterns. In yet another more detailed embodiment, the controller is programmed with executable code configured to enable execution of a further act comprising calculating a standard variation for the historical blood glucose level data within the basal controlled glucose patterns. In still another more detailed embodiment, at least one of the mean deviation and the standard deviation is utilized to establish the suggested insulin dosing regimen for the insulin dosing pump.

In a more detailed embodiment of the fourth aspect, segmenting the historical blood glucose level data into basal controlled glucose patterns and bolus controlled glucose patterns includes identifying a duration that historical blood glucose level data remained above a predetermined threshold, where values at or above the predetermined threshold results in the first person being hyperglycemic. In yet another more detailed embodiment, segmenting the historical blood glucose level data into basal controlled glucose patterns and bolus controlled glucose patterns includes identifying a duration that historical blood glucose level data remained below a predetermined threshold, where values at or below the predetermined threshold results in the first person being hypoglycemic. In a further detailed embodiment, establishing the suggested insulin dosing regimen for the insulin dosing pump includes using an input regarding a type of insulin dosed to the first person. In still a further detailed embodiment, the type of insulin dosed to the first person is at least one of very rapid-acting insulin, short acting insulin, intermediate acting insulin, and long acting insulin. In a more detailed embodiment, establishing the suggested insulin dosing regimen for the insulin dosing pump includes using an input regarding an absorption rate of insulin dosed to the first person. In a more detailed embodiment, the input regarding the absorption rate of insulin is derived from the historical blood glucose level data and the historical insulin dosing data. In another more detailed embodiment, establishing the suggested insulin dosing regimen for the insulin dosing pump includes using an input regarding a physical condition of the first person. In yet another more detailed embodiment, the physical condition of the first person includes an input regarding whether the first person was ill during the first period of time. In still another more detailed embodiment, establishing the suggested insulin dosing regimen for the insulin dosing pump includes establishing a suggested insulin dosing regimen when ill for the first person.

In yet another more detailed embodiment of the fourth aspect, establishing the suggested insulin dosing regimen for the insulin dosing pump includes establishing a suggested insulin dosing regimen when exercising for the first person. In yet another more detailed embodiment, establishing the suggested insulin dosing regimen for the insulin dosing pump includes using an input regarding a type of insulin pump the first person uses. In a further detailed embodiment, the controller is programmed with executable code configured to enable execution of further acts comprising inputting the historical blood glucose level data output from the continuous glucose monitoring device over the first period of time for the first person, and inputting the historical insulin dosing for the first person over the first period of time. In still a further detailed embodiment, the controller is programmed with executable code configured to enable execution of further acts comprising processing additional historical blood glucose level data output from the continuous glucose monitoring device over a second period of time for the first person to segment the additional historical blood glucose level data into additional basal controlled glucose patterns and additional bolus controlled glucose patterns, and using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump. In a more detailed embodiment, the controller is programmed with executable code configured to enable execution of a further act comprising generating a report that includes graphical feedback showing how the additional historical blood glucose level data changes with time. In a more detailed embodiment, the report includes at least one of a mean deviation and a standard deviation of blood glucose rates of change using the additional historical blood glucose level data. In another more detailed embodiment, the controller is programmed with executable code configured to enable execution of a further act comprising generating programming code configured to be executable by a controller of the insulin dosing pump to dose insulin consistent with the suggested insulin dosing regimen. In yet another more detailed embodiment, the controller is programmed with executable code configured to enable execution of further acts comprising processing additional historical blood glucose level data output from the continuous glucose monitoring device over a second period of time for the first person to segment the additional historical blood glucose level data into additional basal controlled glucose patterns and additional bolus controlled glucose patterns, using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump, and generating additional programming code configured to be executable by the controller of the insulin dosing pump to dose insulin consistent with the suggested revised insulin dosing regimen.

It is a fifth aspect of the present invention to provide a blood glucose monitor comprising: (i) a primary housing that at least partially containing a transmitter communicatively coupled to a sensor configured to measure glucose in interstitial fluid, the primary housing at least partially containing a power source in electrical communication with the transmitter and configured to power the transmitter to transmit signals indicative of the glucose measured; (ii) a flexible sensor patch operatively coupled to the primary housing and including an adhesive configured to be applied to the skin of a human; and, (iii) a guard peripherally disposed about the primary housing, the guard including an opening so as not to completely cover the primary housing, the guard including a sloped surface configured to provide a relatively smooth transition between the skin and a top of the primary housing.

In a more detailed embodiment of the fifth aspect, the guard is removably coupled to the primary housing. In yet another more detailed embodiment, the guard is removably coupled to the flexible sensor patch. In a further detailed embodiment, the guard is fixedly coupled to the primary housing. In still a further detailed embodiment, the guard is fixedly coupled to the flexible sensor patch. In a more detailed embodiment, the guard includes a frustoconical shape with an interior cavity configured to receive at least a portion of the primary housing. In a more detailed embodiment, the interior cavity is circumferentially centered and includes a cylindrical shape. In another more detailed embodiment, an adhesive is applied to a base of the frustoconical shape, the adhesive being configured to releasably secure the guard to the skin. In yet another more detailed embodiment, the guard comprises at least one of a thermoplastic and a thermoset. In still another more detailed embodiment, the guard comprises an elastomer.

It is a sixth aspect of the present invention to provide a blood glucose monitor comprising: (i) a primary housing that at least partially containing a transmitter communicatively coupled to a sensor configured to measure glucose in interstitial fluid, the primary housing at least partially containing a power source in electrical communication with the transmitter and configured to power the transmitter to transmit signals indicative of the glucose measured, the primary housing including a peripheral sloped surface with an angle between twenty to seventy degrees with respect to a horizontal plane, the peripherals sloped surface configured to provide a sloped transition between a top of the primary housing and a bottom periphery of the primary housing; and, (ii) a flexible sensor patch operatively coupled to the primary housing and including an adhesive configured to be applied to the skin of a human.

In a more detailed embodiment of the sixth aspect, the primary housing includes a frustoconical shape. In yet another more detailed embodiment, the primary housing comprises at least one of a thermoplastic and a thermoset. In a further detailed embodiment, the primary housing comprises an elastomer.

### BRIEF DESCRIPTION OF THE DRAWINGS

FIG. 1 is a top view of an exemplary diabetic pump.

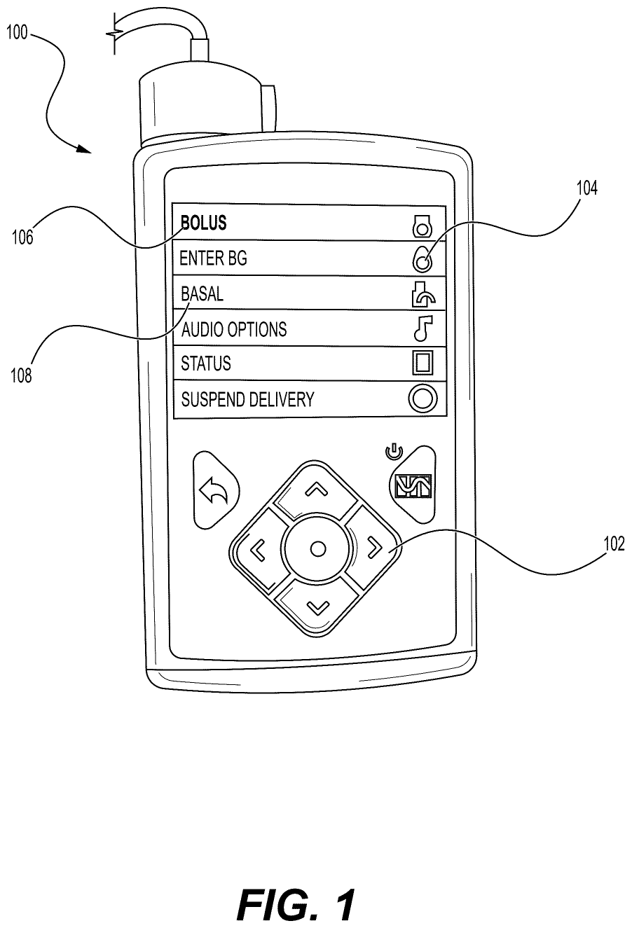

FIG. 2 is multiple top views of the exemplary diabetic pump of FIG. 1, showing a display for use with programming said diabetic pump.

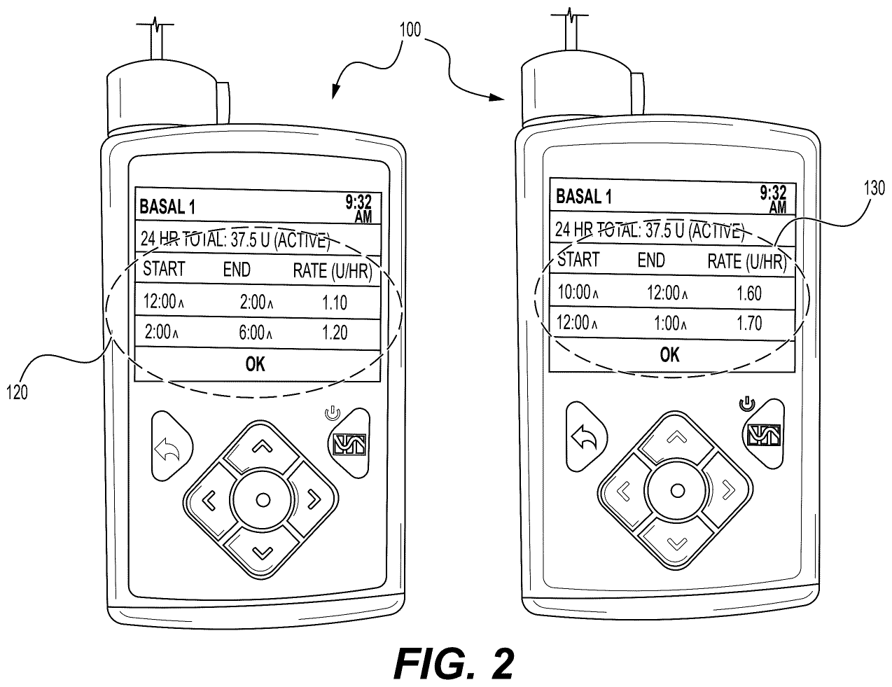

FIG. 3 is a diagram reflecting a blood glucose monitor outputting daily historical glucose records in a graphical form, where this graphical form data can be extracted into a tabular data set.

FIG. 4 is a table representing a tabular form of raw data obtained from a Continuous Glucose Monitoring device for a patient during a period of evaluation.

FIG. 5 is a plot of basal rates programmed into a diabetic pump for a 24-hour duration.

FIG. 6 is a table of basal rates programmed into a diabetic pump for a 24-hour duration.

FIG. 7 is an exemplary diagram depicting a data preprocessing pipeline in accordance with the instant disclosure.

FIG. 8 is a diagram reflecting data conversion from the table of FIG. 6 into a structured dataset in accordance with the instant disclosure.

FIG. 9 is a table reflecting a data log in accordance with the instant disclosure, where the data log reflects how many glucose readings were recorded for each 24-hour duration.

FIG. 10 is a plot of blood glucose readings from a blood glucose monitoring device, as well as a curve approximating the blood glucose readings if continuously taken.

FIG. 11 a plot of blood glucose readings from a blood glucose monitoring device over a 24-hour duration, with curves smoothing the data and showing standard deviation of the data.

FIG. 12 includes a pair of diagrams reflecting blood glucose readings from a blood glucose monitoring device over a 15-hour duration, showing mealtimes for the patient, and instances where the blood glucose readings exceeded a predetermined maximum and fell below a predetermined minimum.

FIG. 13 includes a pair of subset diagrams of FIG. 12, reflecting blood glucose readings from a blood glucose monitoring device over a 15-hour duration, showing mealtimes for the patient, and instances where the blood glucose readings exceeded a predetermined maximum and fell below a predetermined minimum, as well as portions of the blood glucose readings that would be controlled by a bolus dose of insulin.

FIG. 14 is a diagram reflecting smoothed and revised blood glucose monitor records from pattern recognition and addressing irregularities in graphical form, where this graphical form data can be converted into a revised tabular data set.

FIG. 15 is a series of overlaid plots showing blood glucose monitoring data obtained over the course of several 24-hour durations.

FIG. 16 is a histogram visualization illustrating the rate of change of blood glucose over a two-week period for a patient.

FIG. 17 is a control variability grid analysis that tracks maximum and minimum blood glucose readings over a period of several days to evaluate how well the patient's blood sugar is being regulated.

FIG. 18 is a diagram reflecting how insulin is absorbed by a patient's body over time.

FIG. 19 is a diagram reflecting how a series of basal doses of insulin are absorbed by a patient's body over time.

FIG. 20 is a diagram graphically depicting an exemplary process for determining basal rates for an insulin pump in accordance with the instant disclosure.

FIG. 21 is a table showing how optimized basal dosing rates in accordance with the instant disclosure can be modified by certain percentages to output revised basal dosing rates.

FIG. 22 is a series of overlaid plots showing blood glucose monitoring data obtained over the course of several 24-hour durations for a patient having poorly controlled blood glucose levels.

FIG. 23 is a table showing how optimized basal dosing rates in accordance with the instant disclosure can be modified to output revised basal dosing rates in cases where the patient is exercising or is ill.

FIG. 24 is a table showing optimized basal dosing rates for a patient using an insulin pump in accordance with the instant disclosure.

FIG. 25 is a series of blood glucose monitoring data obtained for a patient over a series of 24-hour durations showing relatively good control of blood glucose levels.

FIG. 26 is a series of blood glucose monitoring data obtained for a patient over a series of 24-hour durations where certain portions of the duration have no data and other portions of the durations have data not accurately reflecting blood glucose levels managed just from basal insulin doses.

FIG. 27 is an elevated perspective view of a commercially available blood glucose monitoring device.

FIG. 28 is an elevated perspective view of a portion of a patient's anatomy having the blood glucose monitoring device of FIG. 27 mounted thereto.

FIG. 29 is an elevated perspective view of a portion of a patient's anatomy having the blood glucose monitoring device of FIG. 27 mounted thereto, along with an exemplary sensor guard in accordance with the instant disclosure.

FIG. 30 is a profile view of the blood glucose monitoring device of FIG. 27 having a cross-sectional view of an exemplary sensor guard, in accordance with the instant disclosure, around its periphery.

### DETAILED DESCRIPTION

The exemplary disclosure is described and illustrated below to encompass various solutions to problems faced by diabetics and those overseeing the treatment of diabetes. Of course, it will be apparent to those of ordinary skill in the art that the embodiments discussed below are exemplary in nature and may be reconfigured without departing from the scope and spirit of the present invention. It should be noted, however, that while certain aspects may be described and give the impression that these aspects are independent, it is within the scope of the disclosure to combine one or more of the various aspects disclosed herein to create combinations not expressly indicated as being a combination for purposes of exemplary explanation. However, for clarity and precision, the exemplary embodiments as discussed below may include optional steps, methods, and features that one of ordinary skill should recognize as not being a requisite to fall within the scope of the present invention.

As used herein, the term “computer” is defined herein to mean any device or system capable of performing processing, receiving input from the user or environment or presenting information to the user. And, as used herein, “portable electronic device” means any portable programmable machine capable of executing machine-readable instructions. A portable electronic device may include, but is not limited to, a handheld or portable general purpose computer, microprocessor, microcontroller, digital signal processor, personal computer (PC), personal digital assistant (PDA), laptop computer, notebook computer, smartphone (such as Apple's iPhone™, Motorola's Atrix™ 4G, and Research In Motion's Blackberry™ devices, for example), tablet computer (such as Apple's iPad™, Samsung's Galaxy™, Amazon's Kindle™, and Toshiba's Excite™ devices, for example), netbook computer, portable media player (such as Microsoft's Zune HD™ and Apple's iPod Touch™ devices, for example), wearable computer, point of sale device, or a combination thereof. A portable electronic device may comprise one or more processors, which may comprise part of a single machine or multiple machines.

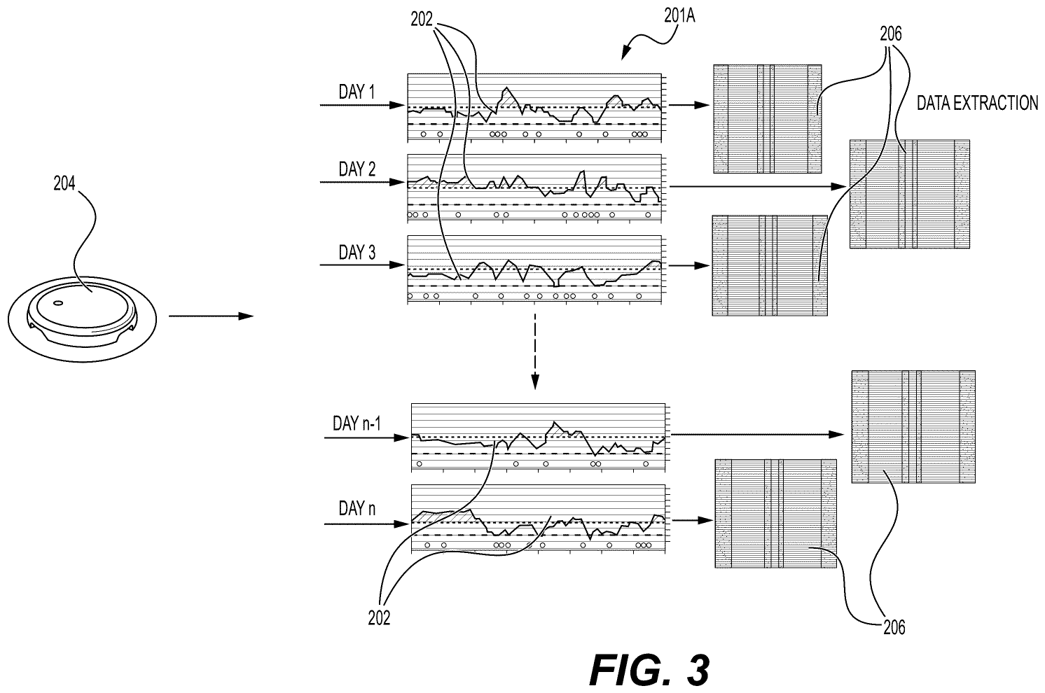

Referencing FIG. 3, the present disclosure includes a computer program that can be implemented with a stand-alone computer or as a mobile application as part of a portable electronic device (which may include a diabetic pump controller to allow a diabetic (user of the diabetic pump) or another person (such as a spouse, nurse, physician, caretaker, etc.) to enter in current or default pump basal rates and glucose data (numerous days' or weeks' worth of data that is patient-specific)) and have the computer program output suggested pump basal rates accounting for the input data. As used herein, the term “patient” refers to a person suffering from diabetes and, optionally, to a person suffering from diabetes that uses an insulin pump to administer insulin to treat his diabetes.

What follows is a detailed description of exemplary technology and an exemplary process for creating personalized (i.e., patient-specific) basal rates for an individual with diabetes with the primary goal, but not necessary to fall within the scope of the disclosure, of maintaining glucose levels within the normal range (euglycemia), which may be between 70-180 mg/dL.

### Step 1: Data Collection

The data collection step aims to collect a personalized extensive dataset, incorporating historical Continuous Glucose Monitoring (CGM) data paired with insulin dosing records. Optional contextual information including health conditions, daily activities, travel leading to changes in altitude, absorption rates (as each person may absorb insulin differently), health condition (healthy vs. having a cold vs. having COVID or the flu, among other illnesses), and various physiological factors can be collected to create different insulin dosage suggestion profiles. For data processing and construction of a computer program model, two separate datasets 201A, 208 may be reconstructed.

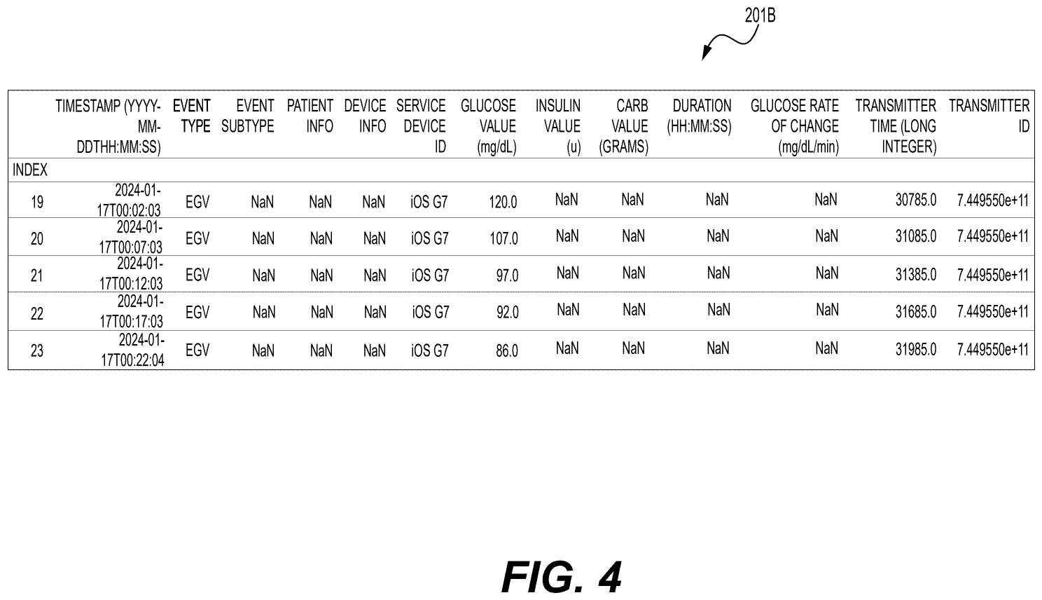

The first dataset 201A and 201B may include recordation of outputs 202 from a CGM sensor/device 204 regarding the patient's blood glucose levels (BGL) over a given period of time. This CGM sensor data can be input to a closed loop computer algorithm to evaluate the high, out of normal range, and low, out of normal range BGL values. Ideally, the present disclosure contemplates having CGM sensor data recorded over the course of many days encompassing various ranges of health conditions and physical activities of the patient. Although this raw CGM sensor data 202 can be output in graphical form, depicted in FIG. 3, this same raw CGM sensor data can also be output into tabular form 206 (FIG. 3) and 201B (FIG. 4) to ascertain the BGL value at a given increment of time (e.g., every five minutes, every fifteen minutes, etc.). This raw CGM sensor data 202 may represent actual readings from a CGM device, depending on the device, every 1-5 minutes, graphically depicted with respect to a 24-hour period. It should be noted, however, that this raw CGM sensor data 202 does not incorporate insulin delivery throughout the day, where sudden rises in BGL may be the result of a patient taking in too much sugar responsive to a low BGL, or a sudden BGL drop because the patient administered too much insulin as a corrective dose. While prior art methods attempt to change basal rates based on raw CGM sensor data 202 generated by a CGM in real-time, the inventors of the instant disclosure have found that taking into account data beyond blood glucose data may aid in predicting appropriate basal rates for a diabetic pump controller.

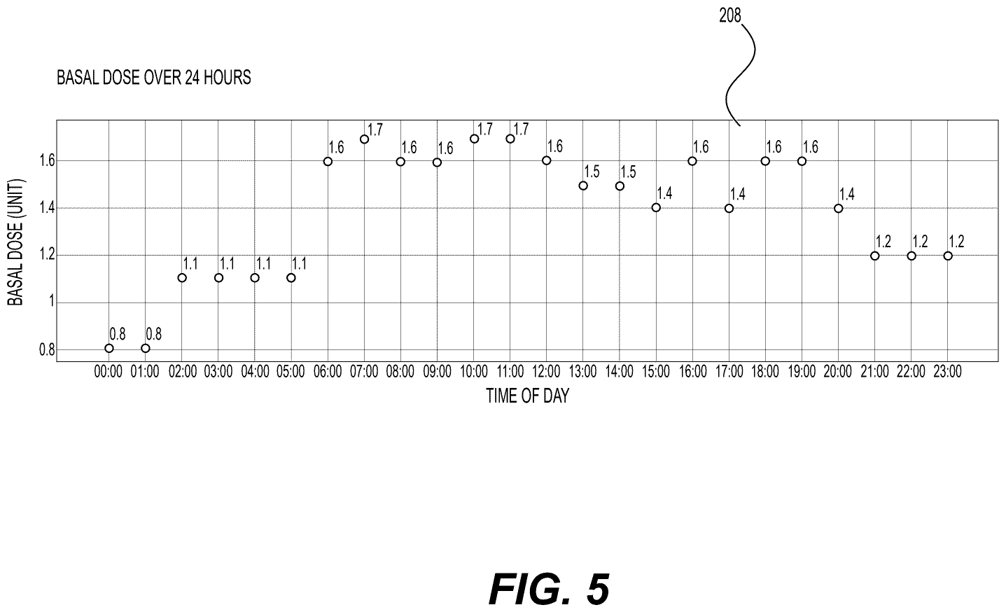

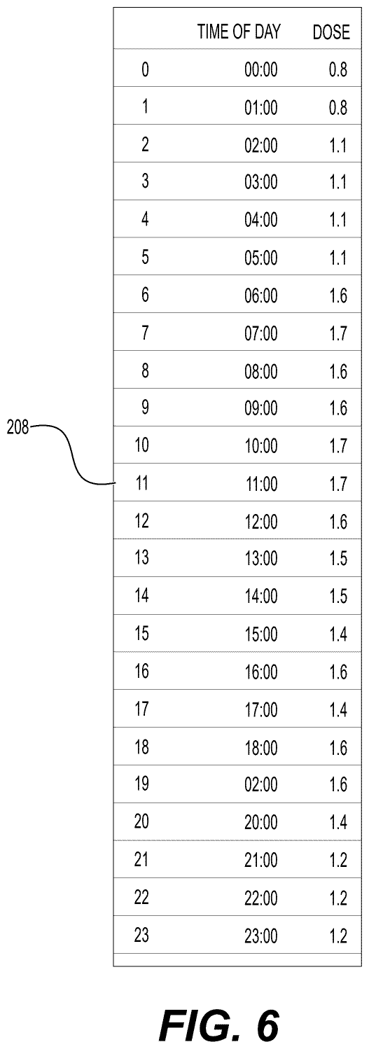

The second dataset 208, as depicted in FIG. 5, may include associated insulin dosing records 208 for that patient during the period of assessment in graphical form or in tabular form as in FIG. 6.

### Step 2: Data Processing and Feature Engineering

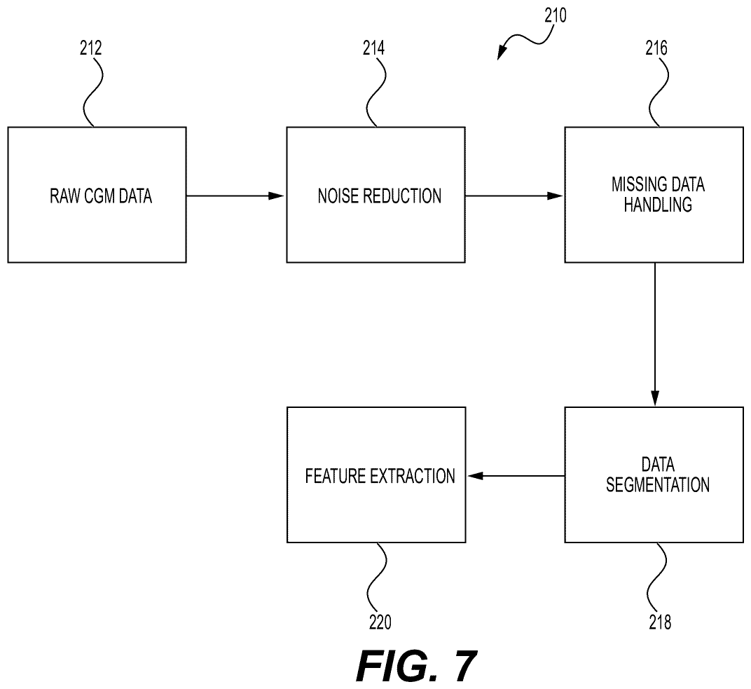

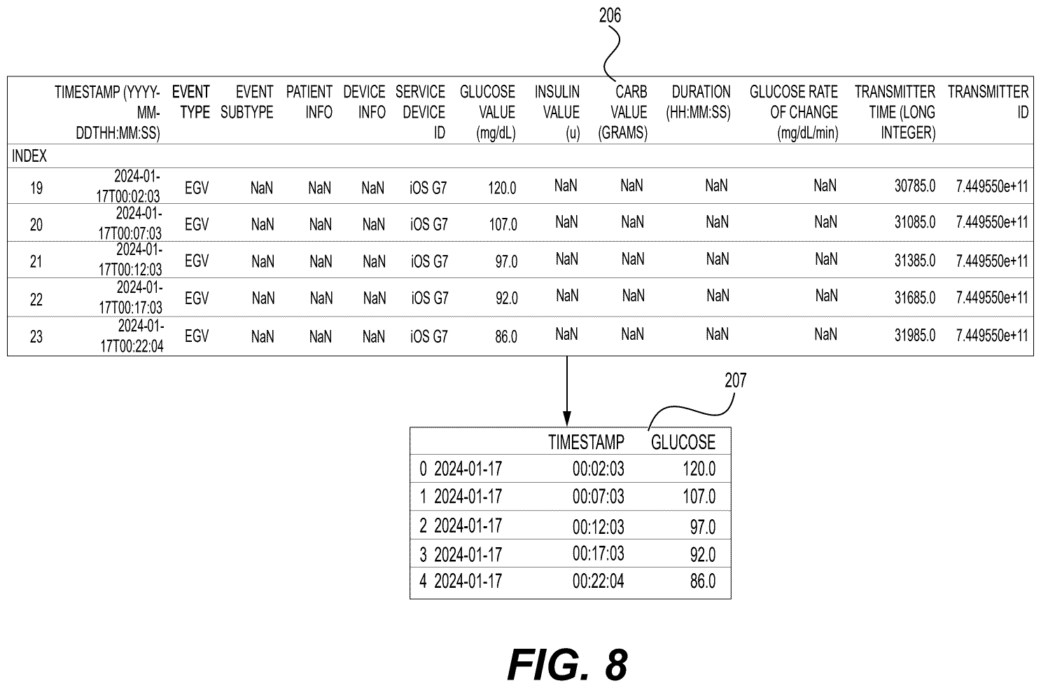

Referring to FIG. 7, an exemplary diagram depicts an exemplary data preprocessing pipeline 210 in accordance with the instant disclosure, where one or more of these steps may be omitted or refined and the pipeline remains functional. Data preprocessing may include transforming raw CGM sensor data 202 from a CGM device 204 as represented by a tabular form dataset 206 to a structured dataset 207, depicted in FIG. 8 suitable for the pre-processing module incorporated into the computer program. This data cleaning process removes unnecessary information from raw CGM sensor data and constructs a tabular form of the reading glucose values and corresponding timestamps. Several techniques may be employed to process raw CGM sensor data 202 from a CGM sensor 204, ensuring smooth and efficient data preprocessing when dealing with large volumes of data, as depicted by the flowchart of FIG. 7.

The exemplary preprocessing pipeline 210 may begin with the step 212 of inputting raw CGM data 202, and may be followed by the steps 214, 216 of noise reduction 216 and handling/addressing missing data 216 at times where the dataset is incomplete from the absence of data generation or where there is an interruption in data reception. Sometimes, the dataset is complete but the timestamps for each day within the assessment period are not consistent. This missing data handling step 216 addresses instances where CGM data 202 is incomplete, missing data points, or inconsistency in timestamps for each day. In exemplary form, a multi-faceted approach may be used to handle missing data that includes identifying the nature of any missing data. By way of example, this multi-faceted approach may distinguish between missing data that occurs randomly and missing data that exhibits a systematic pattern. Randomly missing data may result from sensor errors or other sporadic factors, while systematic patterns could emerge due to sensor calibration issues or device malfunction. Identifying the root cause of missing data may be important for selecting appropriate handling methods.

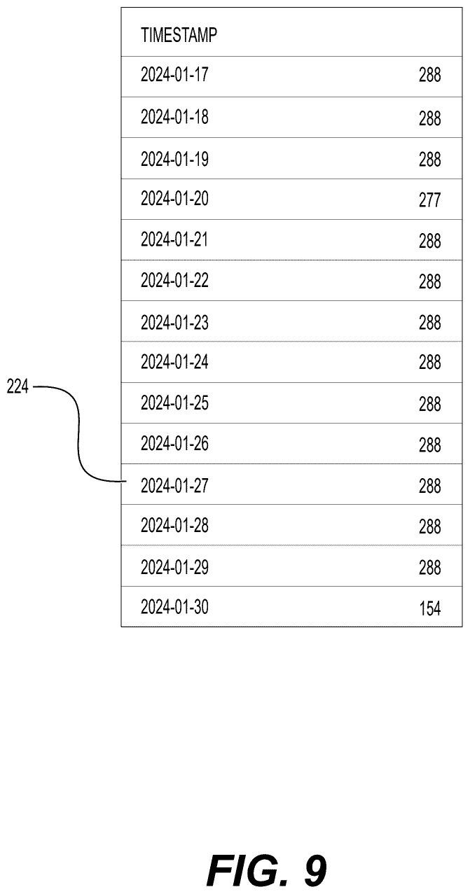

Depending on the type of CGM device used by the patient, each CGM device allows the patient to output glucose data with different timestamps and samples. For example, if the CGM device outputs data every 5 minutes, then a total of 288 data points are exported for one day. If only 5 or 10 data points are missing, it could be due to random issues during data transfer or insufficient contact between the device and the skin. However, if one-third or half of the expected data points are missing for a single day, it may indicate a systematic failure or the patient had to change the sensor. For randomly missing data, linear interpolation may be employed to fill in the missing data, while missing data exhibiting systematic patterns may be handled by identifying and fixing the underlying issue(s) causing these patterns and not attempting to fill in missing data. This may involve sensor recalibration, device maintenance, or addressing environmental factors that affect data collection. For instance, as illustrated in FIG. 9, a data log 224 of the CGM device records how many data points are recorded over a given period, with data points being optionally generated every 5 minutes, resulting in a maximum of 288 data points per day. On 2024 Jan. 20 (corresponding to Jan. 20, 2024, for instance), there were only 277 data points logged, representing approximately a 4% decrease from the expected count. Hence, this loss is considered random. However, on 2024 Jan. 30 (corresponding to Jan. 30, 2024, for instance), only 154 data points were logged, indicating a significant shortfall of about 47%. This suggests a system failure with the CGM device on that particular day, or possibly, the device was removed for maintenance purposes.

Timestamp inconsistencies in CGM data can arise if the device is not regularly maintained or calibrated. Since CGM systems rely on an internal clock, minor drift over time can lead to misaligned or inconsistent recording intervals. This issue is further compounded by factors such as data transmission delays, sensor calibration errors, firmware bugs, or software glitches. When inconsistencies occur, the best course of action is to consult the manufacturer to diagnose and resolve the root cause. However, these irregularities can be effectively managed using interpolation techniques. Since timestamp inconsistencies behave similarly to randomly missing data, methods such as linear and non-linear interpolation can reconstruct missing values while preserving the integrity of the dataset.

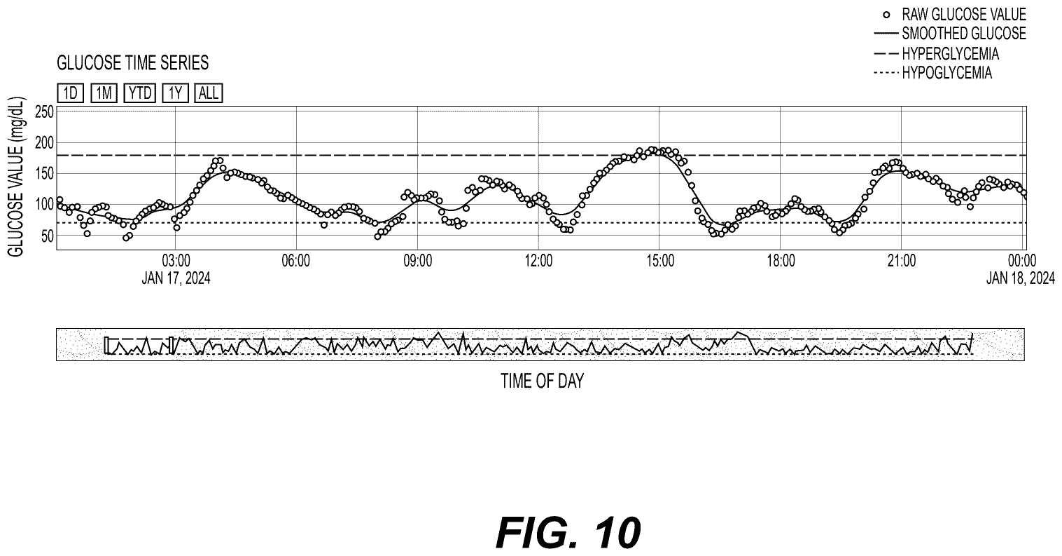

Once noise, missing data, and timestamp inconsistencies are addressed in steps 214, 216, the raw glucose data may undergo a smoothing operation to enhance computational efficiency and data segmentation. The sensitivity of the CGM sensor significantly influences the reading and computation of the glucose level's rate of change. This rate of change can provide valuable insights into meal habits or physical activity. For instance, when a patient consumes a large amount of sugar, the glucose level rises rapidly within a short timeframe, resulting in a sudden spike in the CGM sensor reading. Conversely, when a patient engages in intense physical activity but fails to consume sufficient sugar, it leads to a rapid change in the glucose level. To mitigate these fluctuations, a Locally Weighted Scatterplot Smoothing (Lowess) algorithm may be applied to the raw glucose data. As illustrated in FIG. 10, the dots represent raw glucose values, while the curve tracking the dots depicts the smoothed glucose values.

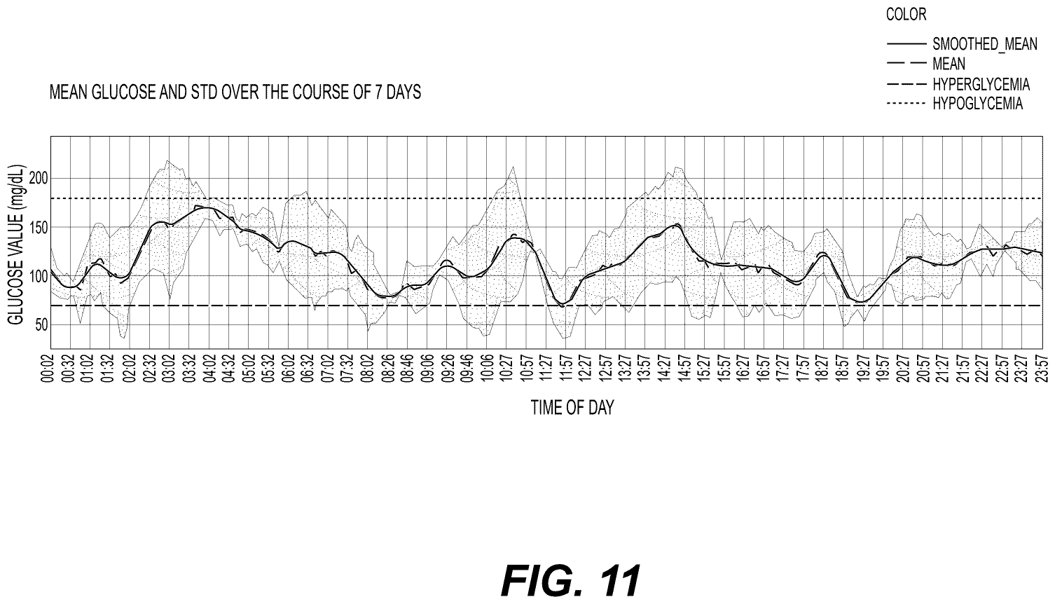

The exemplary preprocessing pipeline 210 may include a step of data segmentation 218 or labeling in order to identify bolus and basal-controlled glucose patterns. Specifically, it distinguishes between patient mealtimes, typically governed by bolus doses, and regular glucose patterns, which are regulated by basal doses. Data segmentation may be applied to the average glucose values during a 24-hour period for the entire pre-recorded dataset, as depicted in the dark blue data points or the red curve representing the best fit function of the data set of FIG. 11, typically spanning one week to one month depending on data availability and the course of assessment.

The rate of change in glucose levels during a meal event is notably higher compared to regular times of the day governed by basal doses. This serves as the primary discriminator for distinguishing meal events from other times of the day. Moreover, meal events exhibit temporal dependencies. Therefore, to identify mealtimes, the Data Assessment Module relies on both the temporal relationships of meal events with the time of day and the rate of change in glucose levels. For instance, breakfast typically occurs between 7:00 am to 9:00 am, lunch between 11:00 am to 2:00 pm, and dinner between 6:00 pm to 8:00 pm. A dedicated algorithm initiates a search from these time windows to identify meal events. If a meal event cannot be located within these windows, the time windows are expanded, and the search is conducted again. In the rare instance where a patient skips a meal and thus a bolus dose is not administered, the algorithm records this occurrence and generates a report for the patient by the end of the week to encourage and maintain healthy habits. Once the glucose data is partitioned into bolus and basal-controlled segments, meal events are removed from the glucose patterns. Within the remaining basal-controlled glucose patterns, they are further segmented into sub-segments based on the acceleration or deceleration of the rate of change in the glucose level. Although the initial algorithm in this disclosure can refer to analysis of basal data, as mentioned above, the more advanced algorithm can address meal times and subsequently bolus doses can be factored into the algorithm and that amount of insulin can be incorporated into the algorithm when determining optimal, personalized basal rates for a patient.

The exemplary process 210 may also include a feature extraction step 220, where relevant features are extracted to characterize different data segments. A first exemplary feature extracted may include statistical features. In this aspect, an array of statistical features that encapsulate central tendencies and variability of CGM data within each segment are generated. These statistical features encompass the mean and standard deviation of the CGM data. A second exemplary feature extracted may include time-domain features. Time-domain features encompass parameters such as the rate of change of BGLs and time above or under specific thresholds or ranges of preferred BGLs. Time-domain features elucidate the temporal aspects of glucose control, allowing the computer program to discern how rapidly glucose levels respond to insulin and the duration of its effect.

### Step 3: Insulin Dosage Suggestions

When making insulin dosage suggestions, several factors may be taken into account, including the type of insulin, the delay in the insulin's effect, the patient's physical condition, and the type of insulin pump being used.

The exemplary process may include the computer program utilizing an input about the type of insulin being utilized by the patient (e.g., rapid acting insulin, short acting insulin, long lasting insulin, etc.) in order to determine the effective working period for the insulin administered. For example, if the patient is using rapid acting insulin, then the computer program will account for the fact that a given bolus dose will work for approximately 4 hours. Once the computer program locates and assesses mealtimes during the 24 period in the BGL pattern, the program may segment these patterns into bolus-controlled glucose 226 and basal-controlled glucose 228. Then, the computer program may allocate rises and the areas under the BGL patterns (from the CGM output data) to the administered bolus dose and to the hourly administered basal dose. In some instances, at mealtime, the patient may not administer enough of a bolus insulin dose and the BGL rises due to consuming a meal and may not come back to a normal level, so the pattern does not represent a parabola, but rather an irregular shape. In such a case, the computer program may assess the initial basal rate before a meal, as well as the rise and the lack of decline of the BGL. Another concern is that glucose activation in the body often occurs much quicker than insulin activation. Therefore, blood sugar levels can initially rise before insulin becomes active. Therefore, the algorithm can take this into account and suggest a higher basal rate 1-2 hours before a meal. Accordingly, the algorithm may store this data and provide a weekly assessment that may include recommendations to the patient that his administered bolus doses are too high or too low at mealtime. The computer program may be programmed to provide these recommendations where a continuous pattern occurs.

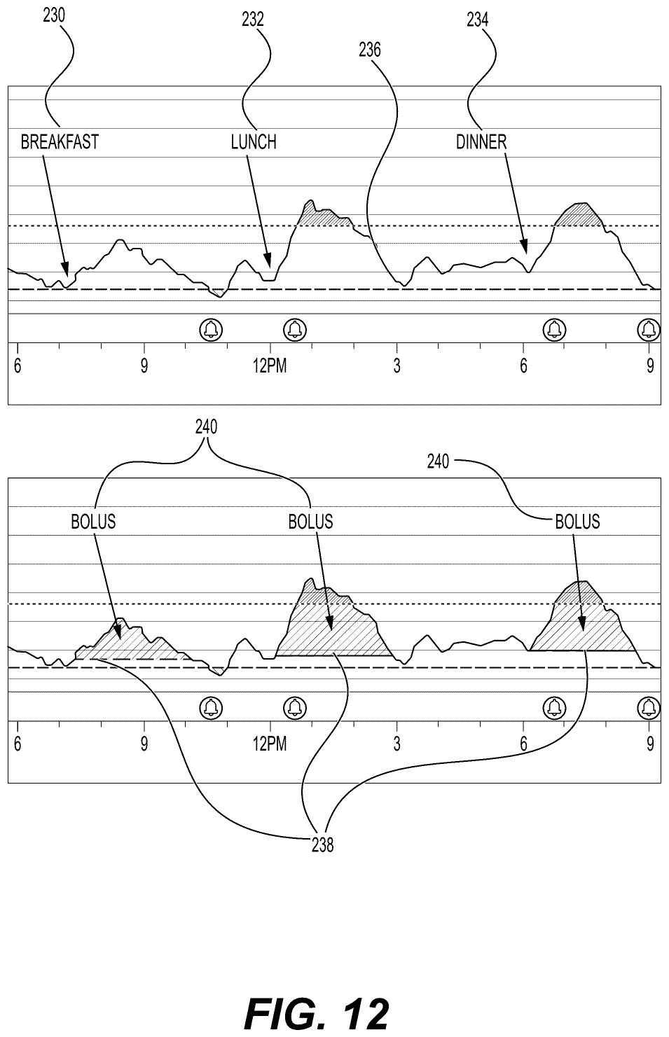

Referencing FIG. 12, after the instant computer program has defined times for breakfast 230, lunch 232, and dinner 234 with respect to the CGM data/plot 236, the computer program determines that BGLs above the horizontal dashed lines 238 at the three mealtimes 230-234 would be controlled using a bolus dose 240 administered by the pump. Every other BGL within the 24-hour period, besides the three mealtime areas, is determined by the computer program to be controlled by the basal administered by the pump using a predetermined regiment. This dashed line could be defined by the algorithm at higher or lower levels depending on the patient. The inventors of the instant disclosure have found that if the computer program were to determine basal rates based off of the raw BGL data in FIG. 3, then there is a high probability that the patient will experience severe BTL lows at mealtimes because the basal rate would be too high so that there would essentially be a double dose of insulin as the patient is also administering a bolus dose before or during mealtime. Accordingly, by using the data processing module discussed above, the computer program is able to identify mealtimes and allocate BGLs between those controlled by a bolus dose of insulin and basal doses of insulin.

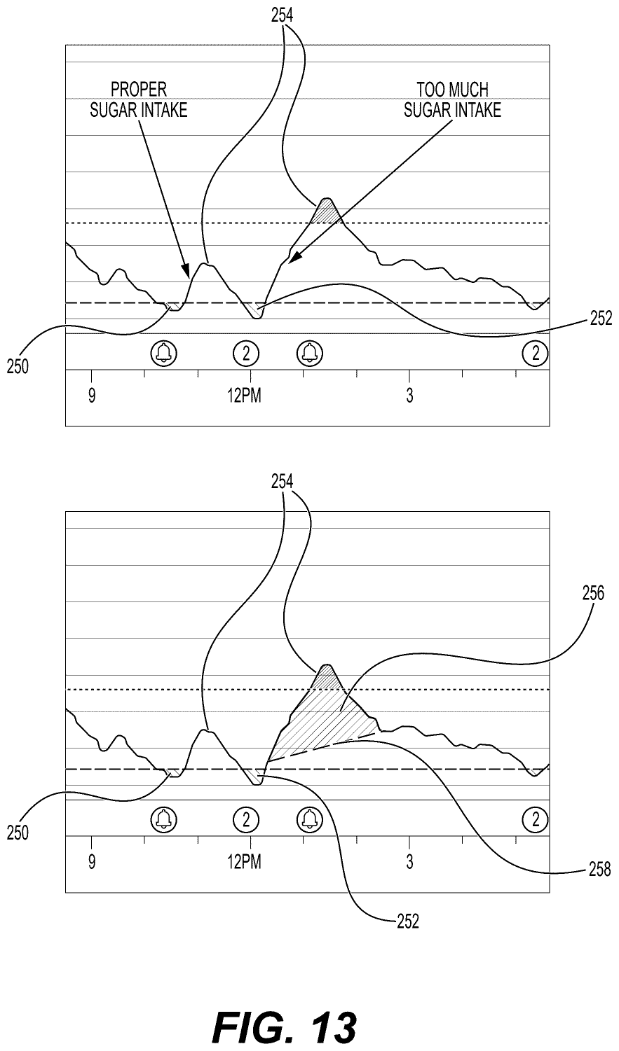

Turning to FIG. 13, at times, a patient will experience a drop in BGLs, which can be a concern because if BGLs go too low it could lead to the patient passing out and potentially leading to a diabetic coma. In exemplary form, the computer program may be operative to use pattern matching or pattern recognition to isolate low BGLs and the patient's response over the next 1-2 hours. During a low glucose occurrence 250, the patient takes in sugar from one or more foods (e.g., orange juice, candy, sugar tablets, etc.) to offset this low BGL. The computer program may identify these low BGLs 250, 252 and may evaluate the patient's bodily response, we well as instances where the patient's BGLs suddenly increase 254 over a short period of time, as well as the bodily response that follows (i.e., changes to the BGL). At times, the patient may administer the correct amount of insulin, but many times the patient will consume insufficient sugar for a given insulin dose, leading to continued low BGL readings, or too much sugar leading to a sudden rise in BGLs. In instances where there is a sudden rise in BGLs, what typically follows is a patient administered bolus dose of insulin, in an attempt to lower the BGLs within a normal range. Therefore, when sudden rises in BGLs occur, is not a normal occurrence controlled by a basal rate, and the computer program recognizes these occurrences as BGLs that are controlled by one or more bolus doses 256 and the computer program accounts for this bolus dosage by not overcorrecting the basal dosage.

In FIG. 13, two different low glucose levels 250, 252 are depicted. The first occurrence 250 was controlled by a proper amount of sugar being administered by the patient, but the second occurrence 252 leads to the patient consuming too much sugar. This second occurrence 252 often occurs when a patient experiences a second sudden drop in BGLs leading to a bit of fear and doubt causing the patient to take in too much sugar to overcompensate for fears of BGLs going lower. When this overcompensation occurs, the computer program does not utilize the BGLs above the dotted line 258 to calculate the optimal basal rate during that time because the BGLs above the dotted line will be controlled by the corrective bolus 256 administered by the patient. It should be noted that the computer program need not recognize this second occurrence 252 as a regular occurrence so that the computer program will be expecting BGLs of this variance to happen every day or nearly every day at a given time. Instead, the computer program may be configured to recognize this second occurrence 252 as an abnormality and treat it as such.

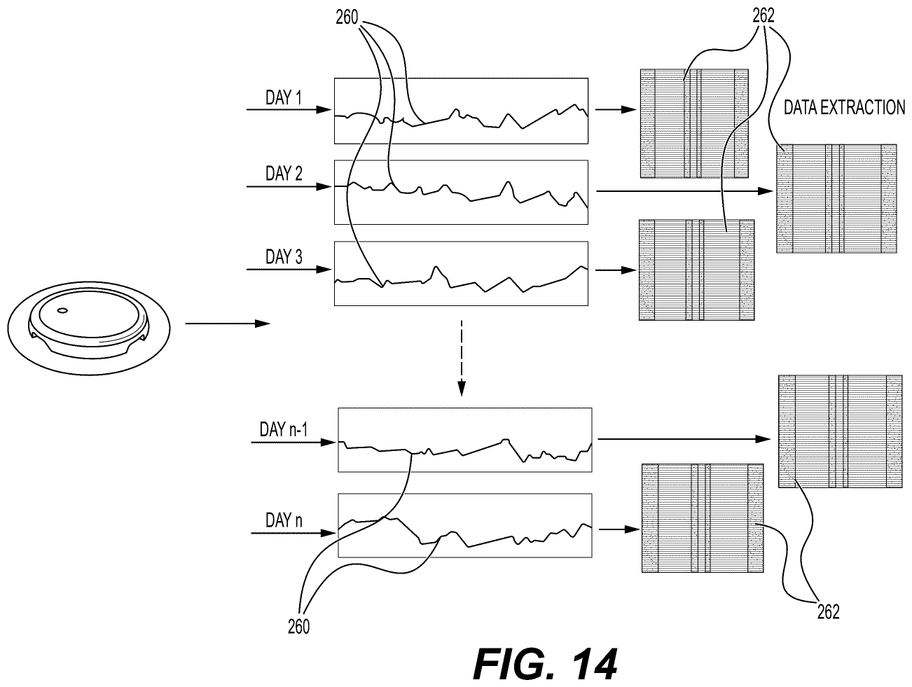

With reference to FIG. 14, BGL patterns and irregularities depicted in FIGS. 12 and 13 may be understood and isolated by the computer program, which may include a smart algorithm that continues to process data and learns, while adding information to the database to optimize the pattern recognition and matching capabilities. A possible output of the instant disclosure is to predict/define basal rates for the patient excluding contributions from bolus doses throughout the day. Once pattern recognition is defined by the computer program, the plots 202 in FIG. 3 can be refined 260 as shown in FIG. 14, so that sudden rises in BGLs are removed and replaced with normal interpolated BGL data as if the BGL was being controlled by basal doses throughout the interpolated period. The revised plots are then utilized by the computer program to generate tabulated revised BGLs 262.

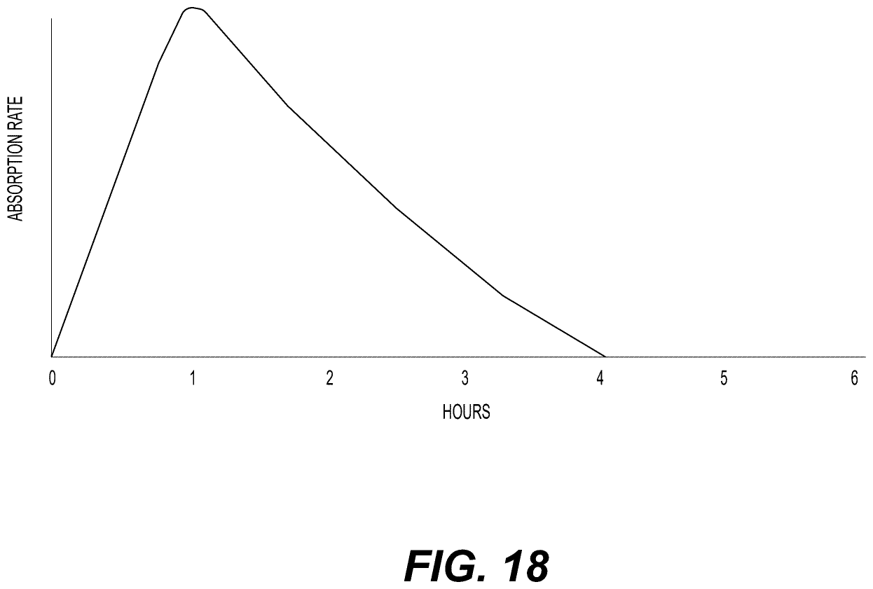

Insulin sensitivity may include how sensitive a patient is to insulin, which can vary widely from patient to patient. Some patients may require more insulin to lower BGLs, while others may need less. In addition, when administered, the same dose of insulin may be utilized by patients at different rates. For example, Humalog is a fast-acting insulin that starts to work within 15 minutes after an injection and peaks in about 1 hour. Humalog continues to work for about 2 to 4 hours and works by lowering levels of glucose (sugar) in the blood. NovoLog is a man-made insulin used to control high blood sugar in adults and children patients. Like Humalog, NovoLog is a rapid-acting insulin that helps lower mealtime blood sugar spikes. Both Humalog and NovoLog are reported to peak absorption at 1 hour after insulin dose administration and continue working for four hours, as shown graphically in FIG. 18, which may be representative of manufacturer absorption information. Unfortunately, it has been discovered that peak absorption for Humalog and NovoLog can occur between 1.5 to 2.0 hours after dose administration and can last in excess of 4 hours. Thus, the absorption pattern advertised by insulin manufacturers may not be representative of the actual insulin absorption rate/pattern of every patient.

Since insulin absorbs in a patient's body over a long period of time, a corrective insulin dose (bolus) will never bring high BGLs back to a normal level immediately. In fact, lowering of BGLs that were very high may actually lead to low BGLs because of follow-up bolus doses that over correct for high BGLs. Therefore, even if a patient uses a closed-loop controlled insulin pump where a sensor gives feedback to the pump to administer a basal or bolus dose, this will result in a 4-hour delay before all of the insulin from this administered dose will be fully absorbed by the patient's body. With this in mind, determining proper basal rates for a patient should include data regarding a patient's dose absorption pattern and the time it takes for insulin to be fully absorbed in the patient's body.

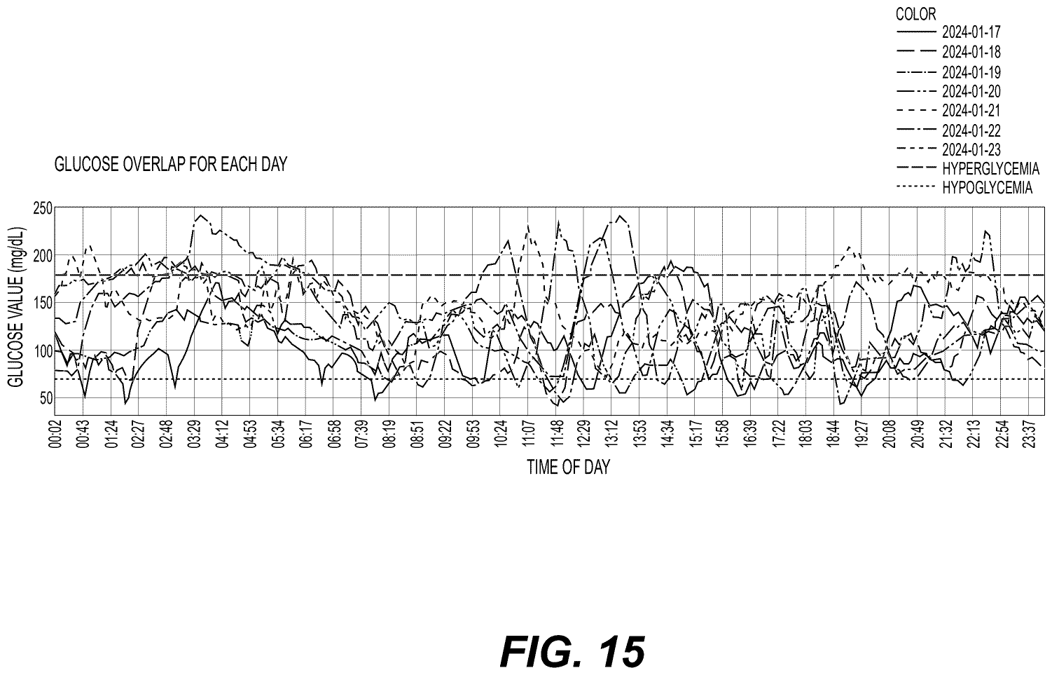

It's important to recognize that glucose levels fluctuate significantly from day to day. Relying solely on glucose values from a single day to adjust basal rates is not recommended. Instead, it's advised that basal rates may be reassessed based on at least one week of evaluation for new patients who have not yet benefited from the present disclosure, and two weeks or more for patients who have been following the guidelines outlined per the present disclosure. As depicted in FIG. 15, glucose values vary greatly each day over the course of a week. Not only do the glucose values fluctuate, but the temporal shifts each day can also influence judgments if based solely on one day's data to revise basal rates. However, analyzing the trends and tendencies of glucose fluctuations over one or two weeks (FIG. 11) can provide a more informed basis for adjusting basal rates. When revising basal rates, it may be important to consider situations where the patient may not be receiving adequate basal doses. For example, as illustrated in FIG. 11, from 2:00 am to 4:00 am, the glucose level rises while the patient is asleep. The algorithm detects this anomaly in the glucose pattern, and this abnormality may be factored into the basal dosing suggestion by increasing the basal rates during sleep time considering the delay in the insulin absorption.

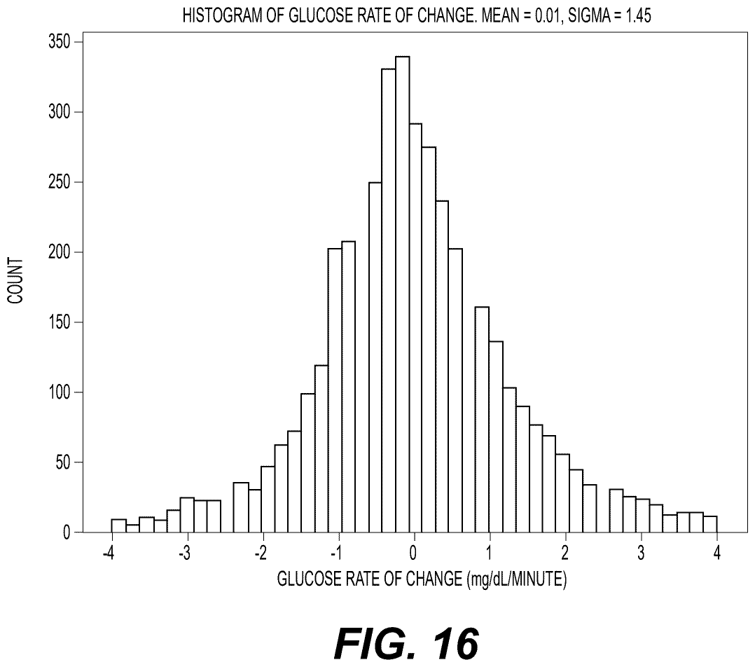

As mentioned earlier, the algorithm examines the rate of change in glucose levels, particularly focusing on the smoothed mean glucose levels over an assessment period, which may span one or more weeks (such as 4-6 weeks). The standard deviation of the rate of change in glucose levels serves as an indicator of glucose sensitivity. A higher standard deviation suggests increased variability in BGLs, indicating poorer control over glucose levels. An effective method to enhance glucose management over the assessment period is for the patient to compare the standard deviation between treatment changes (i.e., changes in the basal rates programmed into the insulin pump versus actual BGLs). For instance, after one week of implementing a treatment change, the algorithm in accordance with the instant disclosure may generate an assessment report containing various useful information, including the standard deviation of the rate of change in glucose levels. The patient can then compare the standard deviation of the current week to that of the previous week. A smaller standard deviation indicates successful implementation of good glucose management practices. As depicted in FIG. 16, a histogram visualization illustrates the rate of change over the course of a two-week assessment period. Sigma value represents the standard deviation of the rate of change in the glucose level.

The rate of change in the glucose level is mathematically defined as follows:

$$rate of change [ ( mg / dL ) / min ] = \frac{{GV}_{{t}_{i}} - {GV}_{{t}_{i - 1}}}{{t}_{i} - {t}_{i - 1}}$$

Where rate of change is expressed in units of (mg/dL)/min.
GVₜ_{ᵢ}: Glucose value at time tᵢ
GVₜ_{_{i−1}}: Glucose value at time tᵢ₋₁
tᵢ and tᵢ₋₁ at two consecutive times where the glucose values are used to calculate the rate of change.
   When calculating the rate of change in glucose levels, smoothed mean glucose values may be utilized, as they may yield better and more reliable results compared to using raw mean glucose values.

The algorithm in accordance with the instant disclosure may rely on the sign of the rate of change to execute adjustments and recommend basal doses. As per the equation provided, the rate of change value can be either positive or negative. A positive sign indicates an increase in glucose levels. In such cases, the algorithm adjusts the basal rates by increasing the doses to counteract this in glucose levels. Conversely, when the sign is negative, indicating a decrease in glucose levels, the algorithm decreases the doses to accommodate for this decline.

The amount of basal dose adjustment may depend on the features extracted during the feature extraction step 220 (see FIG. 7) discussed herein. The basal rate may vary depending on the insulin pump used by the patient. For instance, if the basal rate can be programmed for a 1-hour period, 24 basal doses are calculated for 24 hours, with each hour assigned a basal rate. The relationship between the current basal rates and the suggested basal rates is as follows:

$${r}_{i} = {r}_{ci} + {a}_{i} ( {μ}_{i} + {b}_{i} {σ}_{i} ) + {c}_{i} R o {C}_{i} - {d}_{i} {τ}_{i}$$

### Where:

rᵢ is a new basal rate at i o'clock
r_{ci} is the current basal rate at i o'clock
aᵢ is the weighted factor of the statistic feature from i to i+1 o'clock
uᵢ is the mean glucose value from i to i+1 o'clock
σᵢ is the standard deviation of the glucose values from i to i+1 o'clock
bᵢ is the weighted factor of the σᵢ at i o'clock
cᵢ is the weighted factor of the average rate of change values from i to i+1 o'clock
RoCᵢ is the average rate of change from i to i+1 o'clock
dᵢ is the weighted factor of the delay in insulin absorption at i o'clock
τᵢ is the insulin absorption delay at i o'clock

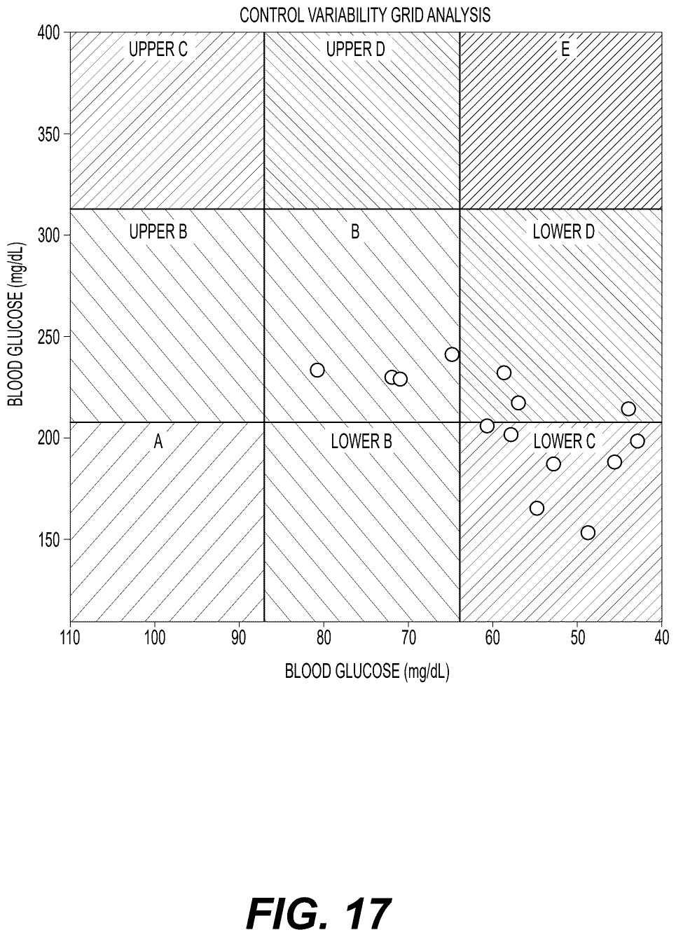

Once the patient receives the revised basal rates and implements the recommended basal doses, a weekly assessment report may be sent to the patient to track glucose management and basal dose adjustments. This report may include a chart illustrating overall glycemic control throughout the assessment period. Known as Control Variability Grid Analysis (CVGA), see FIG. 17, this chart offers insightful information regarding blood glucose control. Each dot on the CVGA represents one day within the assessment period. The horizontal axis of each dot represents the minimum glucose value for that day, while the vertical axis represents the maximum glucose value for that day. Ideally, a well-managed glucose day falls within the green areas of the graph, with the optimal outcome being in the “A” subsection.

Insulin pumps usually administer insulin doses every 5 minutes, even though a basal rate may be set for a 1-hour period. As part of the control of most insulin pumps, a series of basal rates are utilized, which may be preprogrammed or selectively input by a patient or healthcare provider. It is not uncommon for insulin pumps to be programmed with 1-5 basal rates that change during the day. But it has been determined that simply using 1-5 basal rates throughout a 24-hour period is not the best way to control insulin basal doses. With some insulin pumps, patients can enter as many basal rates as desired, but it is not uncommon for a patient to program his pump with a basal rate for each hour of a 24-hour period. Nevertheless, a programmed hourly basal rate for an insulin pump may be divided equally in twelve evenly spaced doses administered every five minutes. This relation can be described using the following expression:

$$ABD = BPR / BO$$

where ABD refers to the basal dose administered by the insulin pump, BPR refers to the basal rate programmed for the pump for a given period, and BO refers to the number of basal doses delivered by the pump within the given period. For example, if the BPR for a 1-hour period is 1.2 units of insulin and the pump administers insulin every five minutes (BO=12), then the ABD would be 1.2/12 or 0.1. In this example, the insulin pump would be programmed to 0.1 units of insulin every five minutes for a given 1-hour duration.

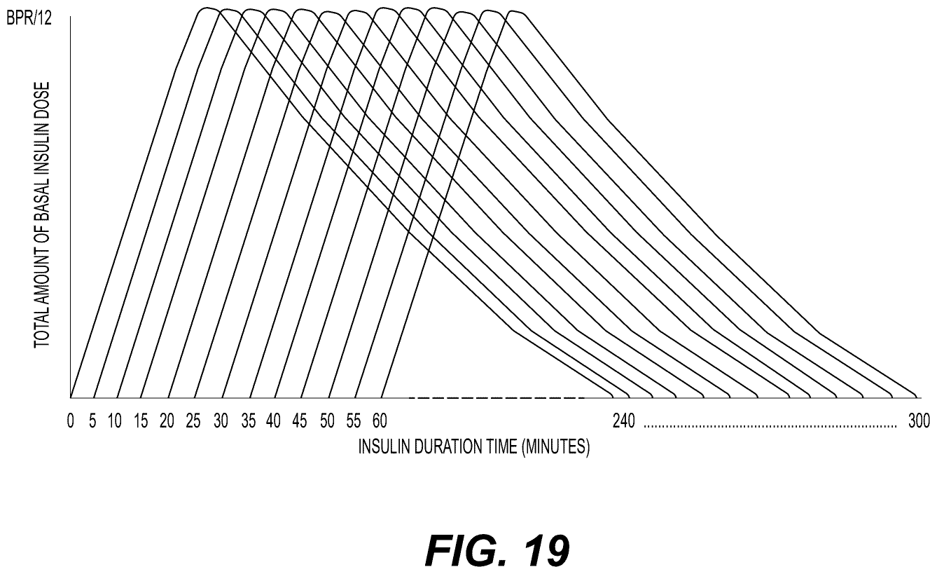

Referring to FIG. 19, sequential and periodic basal dosages administered by an insulin pump during a 1-hour period will actually be absorbed by the body using the pattern shown. If the actual insulin dose is 1.2 units for a 1-hour period, then the insulin pump will administer 0.1 units of insulin every 5 minutes if the specific pump administers basal doses 12 times per hour, but each insulin dose is not absorbed fully by the patient within this 1-hour period. Rather, the insulin administered over this 1-hour period is really absorbed over a 4-hour period. More specifically, in this example, while the insulin pump provides the patient with 1.2 units of insulin during this 1-hour period, the patient is not actually absorbing and using the entire 1.2 units within the same 1-hour period. As a result, the basal rates upstream and downstream need to account for the latency of insulin administered to a patient but not yet absorbed.

In accordance with the instant disclosure, a parametric function is created by the computer program model to approximate the absorption of insulin, which is patient specific, to approximate the absorption rates depicted in FIG. 19. By way of further example, the instant disclosure may make use of this function to arrive at an interval administered basal dose (IABD) using this parametric function, the programmed basal rate, and the number of basal doses as follows:

$$IABD = ( BPR / BO ) * ( {a}_{n} - {x}^{n} + {a}_{n - 1} {x}^{n - 1} + … + {a}_{2} {x}^{2} + {a}_{1} x + {a}_{0} )$$

In addition to understanding absorption rates of a patient, one should also know how much insulin a patient requires to process a particular amount of glucose. For example, during a bolus administered insulin dose that occurs at mealtime, it has been determined that a patient will require 1 unit of insulin for every 20 mg/dL of glucose. Likewise, for example, it has been determined that for this same patient, a basal administered 1-unit dose of insulin is needed for every 30 mg/dL of glucose. It should also be noted that these rates may vary during exercise and illness. Also, this rate will vary for each patient and again, this rate may change as a person ages.

The exemplary computer program model may be programmed to determine optimal basal rates of insulin to account for bodily changes of the patient when exercising or falling ill. When a diabetic exercises, the required amount of insulin administered as a basal rate is much higher than under normal conditions. Conversely, when a diabetic is ill, the amount of insulin administered as a basal rate is much lower than under normal conditions. Generally, the more severe the illness, the more insulin will be required for a given amount of glucose. Accordingly, the instant computer program model may account for these insulin variances by taking into account the wellness of the patient and amount of physical activity the patient is undertaking or intends to undertake.

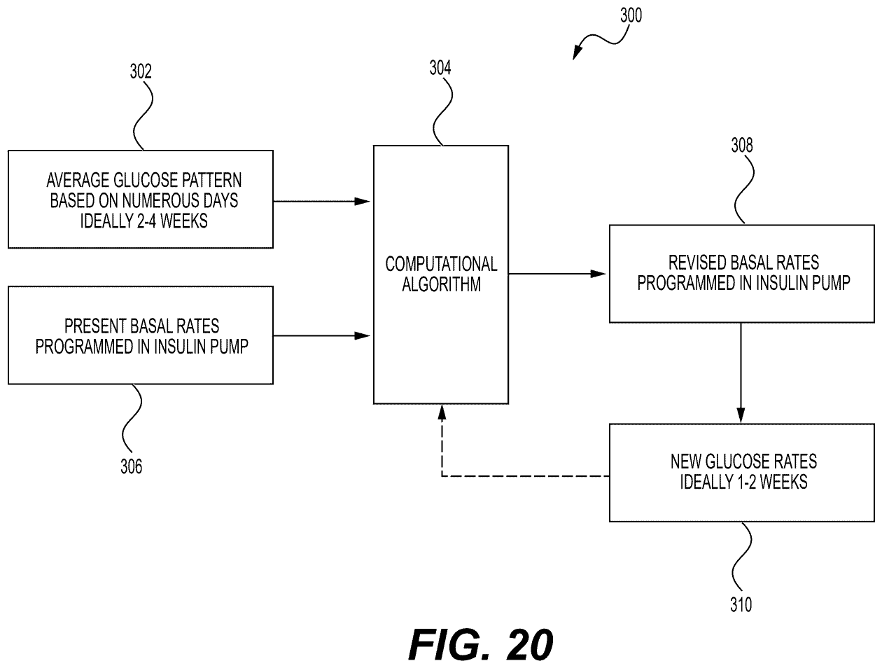

Referencing FIG. 20, an exemplary process 300 in accordance with the instant disclosure for determining basal rates for an insulin pump may utilize CGM data for a lengthy period of time (e.g., 2-4 weeks) 302. This CGM data may be output at predetermined intervals or may be output at intervals that vary. In exemplary form, the CGM data may have CGM data removed where the changes in glucose are attributable to bolus doses and/or variances in insulin absorption patterns.

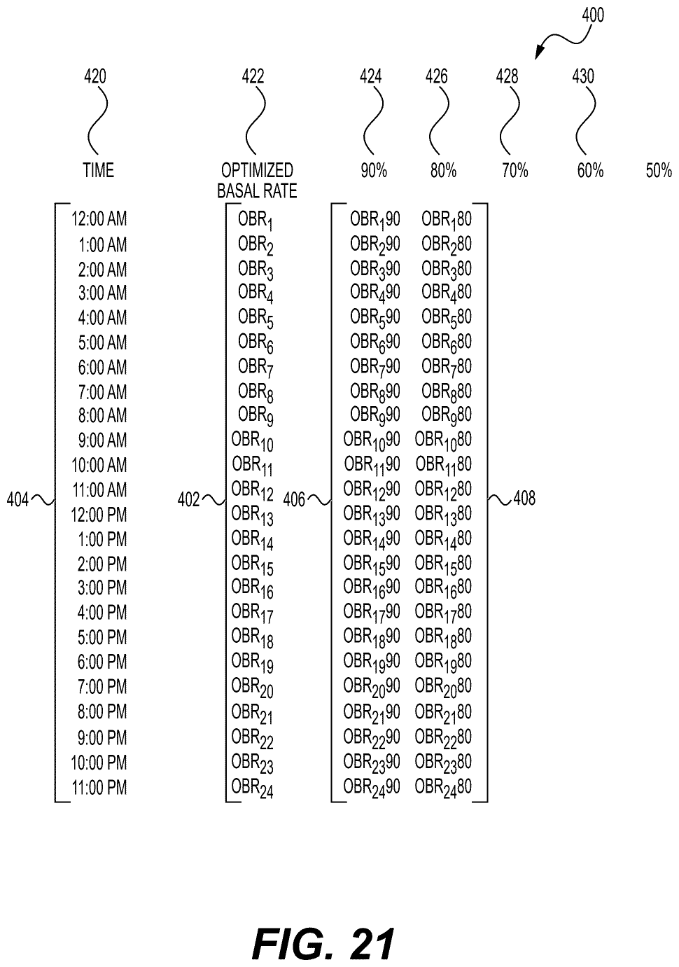

Referring to FIGS. 20 and 21, by way of example, the computer program model may utilize pattern matching and recognition to identify and differentiate CGM data being attributable to basal insulin doses versus bolus insulin doses. This basal CGM data is fed to an algorithm for processing 304, in addition to feeding a patient's current or default basal rates 306 programmed for his insulin pump. The algorithm may then output revised basal rates 308 for the insulin pump, which may comprise periodic rates for each day for between 1 and 14 days, for example. Greater duration may be achieved if desired. And the process may be augmented by the input 310 of new glucose rates as further historical data is generated.

In particular, the computer program model may generate a table 400 that provides suggested basal rates 402 for a given period of time 404, as well as percentages 406, 408 of the suggested basal rates for those same given periods of time. By way of example, FIG. 21 comprises a table 400 with a first column 420 comprising twenty-four hour increments (corresponding to each hour of a calendar day) 404, and a second column 422 providing the basal rate 402 the computer program model suggests for that hourly increment. In addition, the table may include one or more additional columns 426-430 that include fixed percentages of the basal rates appearing in the second column 422. By way of further example, the third column 424 may comprise basal rates that are, for each increment of time, ninety percent of the suggested basal rate 402 of the second column 422. Likewise, the table may include a fourth column 426 that may comprise basal rates that are, for each increment of time, eighty percent of the suggested basal rate 402 of the second column 422. Additional columns 428, 430 may be added to the table to provide corresponding basal rates that are a predetermined percentage of the suggested basal rates 402 of the second column 422. For example, additional columns may include suggested basal rates that are greater than one hundred percent of the suggested basal rates of the second column 422. Although the depicted columns are percentages of the suggested basal rates 402 listed in the second column 422, the basal rates in further columns can start at any percentage value and the increments can be of any amount. The reason these percentage rates are shown is directly related to how well a person has been taking care of themselves with diabetes and how well this person's initial basal rates are controlling his diabetes.

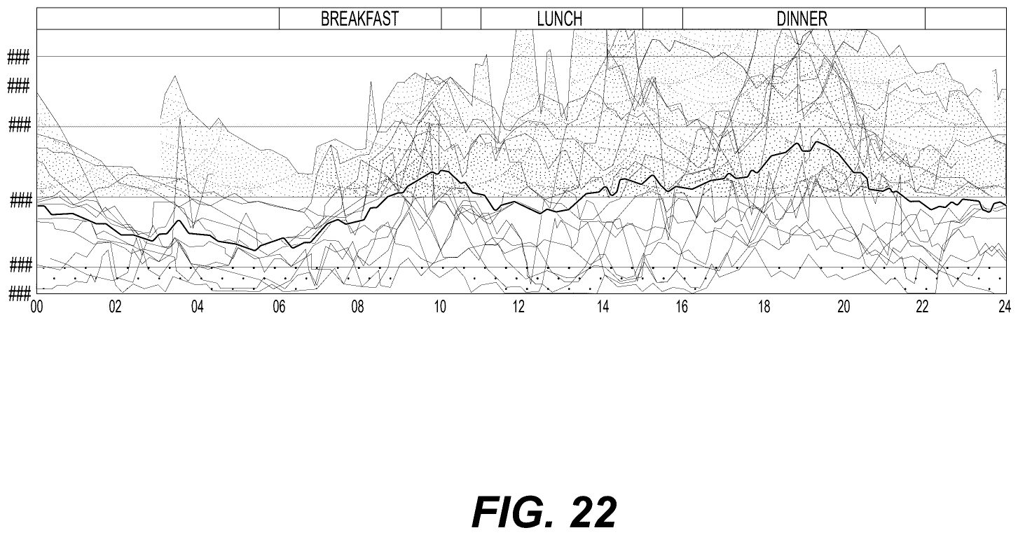

Turning to FIG. 22, if a person is having difficulty controlling his glucose levels, it would not be preferred to use the suggested basal rates of the second column of FIG. 21 because the person may have an actual glucose level that is significantly higher each hour than what is presumed by the computer program model. Accordingly, it may be more difficult to control and correct a person's basal rates using incremental basal dosages, where these corrections may lead to higher basal dosages and overcorrections that may lead to excessive insulin and ultimately low blood sugar levels. Therefore, by way of example, it is envisioned that for a person with uncontrolled glucose levels, one may start using lower percentages of the suggested basal rates (e.g., say 50% or 60% of the optimized basal rate 422) in order to get better control over the glucose fluctuations and, thereafter, after the blood glucose patterns become more normalized or better corrected, transition this person to one or more higher percentage suggested basal rates, such as those of columns 422-426 of FIG. 21. By way of example, during this implementation, the computer program model may optimize the amount of bolus needed by the person just prior to mealtime.

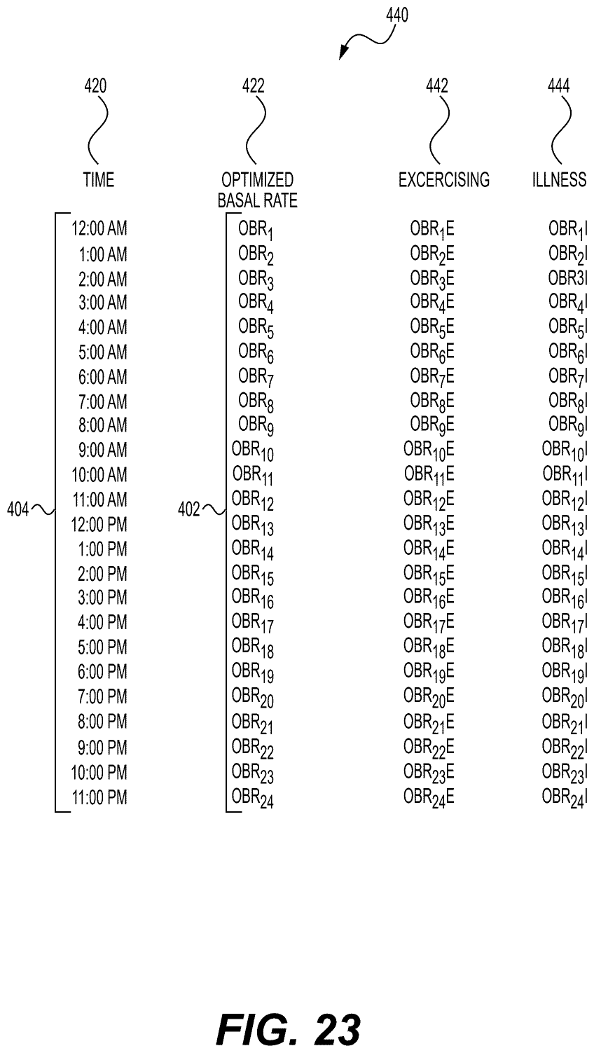

Referring to FIG. 23, as previously introduced, insulin absorption changes dramatically if a person is exercising or is ill. Therefore, the computer program model may include a module that assesses this information for an individual person with diabetes and may generate a table 440 that lists basal rates in case the person is exercising or is ill. By way of example, the table includes the same first and second columns as the table in FIG. 21 and a discussion of these aspects will not be repeated in furtherance of brevity. But this table 440 includes a third column 442 and a fourth column 444, which may be generated by the computer program model in order to generated suggested basal rates for a person while the person is exercising or while the person is ill. Those skilled in the art familiar with insulin pumps understand that many insulin pumps allow for implementation of alternative insulin basal rate dosing regimens, where each alternative insulin basal rate dosing regimen is saved in memory as part of the insulin pump.

Consequently, the instant disclosure allows the computer program model to generate one or more suggested basal rate dosing regimens, where the dosing regimens may include basal rates specific to when the person is exercising or is ill. In order to provide these suggested insulin basal rates, the instant computer program model may obtain CGM data associated with the particular person while exercising or being ill. For example, the instant computer program model may obtain CGM data specific to illnesses such as, without limitation, a cold, influenza, and COVID-19. Moreover, the instant computer program model may obtain CGM data specific to an exercise activity such as, without limitation, basketball, soccer, tennis, swimming, and golf. And using this CGM data, the computer program model can generate one or more suggested basal rate dosing regimens.

Accordingly, a person can program one or more suggested basal rates in their pump as an alternative regimen, where each regimen may be unique to a type of exercise (e.g., golfing, basketball, tennis, etc.) or may be unique to a type of illness (e.g., cold, influenza, COVID-19, etc.). Thus, when the person is exercising, the person can have the insulin pump follow a different, preprogrammed basal dosage rate regimen that may be specific to that type of activity or general increase in activity. Moreover, when the person is ill, the person can have the insulin pump follow a different, preprogrammed basal dosage rate regimen may be specific to that type of illness or general lessening of body activity. By way of example, it is envisioned that proportionally higher rates of basal insulin dosage will be in order for a patient exercising when compared to lesser normal activity levels, whereas it is envisioned that proportionally lower rates of basal insulin dosage will be in order for a patient that is ill when compared to greater normal activity levels.

The computer program model may process data beyond CGM data in order to output suggested insulin basal dosage rates, whether those rates are specific to exercising, specific to illness, or specific to how well controlled the person's glucose levels are. For example, this additional data may include, without limitation, time duration for an exercise, time durations a person is not readily moving, instances where a person forgot to administer a bolus dose of insulin prior to or during a meal, instances where the insulin pump did not administer insulin because there was a blockage or possibly air bubbles in administration tube, instances when the insulin pump was set to suspend and, therefore, was not administering insulin, and instances where there was a CGM sensor error.

The exemplary computer program can also become more detailed, allowing patients to log their daily activities, health conditions, and various physiological factors, creating a comprehensive health journal. These user inputs are directly linked to the suggested insulin basal dosage rates, allowing for personalized insulin management. Over time, as patients consistently provide these inputs, the system builds a historical record of tailored insulin dosage recommendations, effectively generating multiple insulin profiles. These profiles serve as a valuable reference, allowing patients to select the most suitable insulin regimen for a given week, particularly when there are significant changes in their routine, such as increased physical activity, illness, or dietary adjustments. This adaptive approach helps maintain optimal glycemic control while accommodating lifestyle variations. The patient could also input travel on an airplane which would lead to altitude changes that could affect insulin absorption, time zone changes, sleep patterns, which change during travel, and fatigue due to travel.

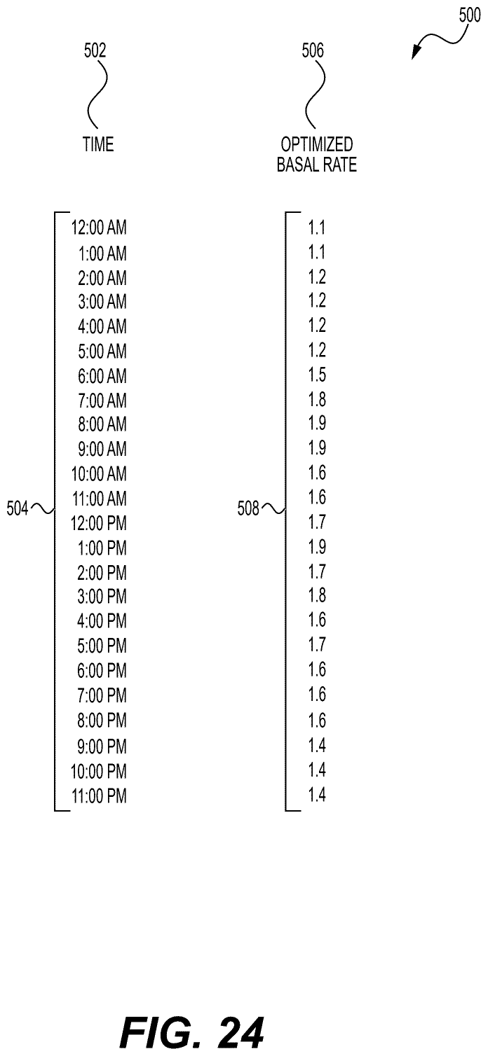

With reference to FIG. 24, the computer program model may generate a table 500 that includes a first column 502 specific to intervals of time 504 throughout a twenty-four hour period. By way of example, these intervals may be one hour or some fraction of an hour. One-hour intervals are shown only for purposes of illustration in the table 500. Accordingly, it is within the scope of the disclosure for the computer program model to generate suggested insulin basal dosage rates for any predetermined period of time, including per minute, per hour, or per predetermined minute increment. The table 500 also includes a second column 506 with suggested basal insulin dosage rates 508, where 1.1 in the table refers to a dosage rate of 1.1 units of insulin per hour. For purposes of the instant disclosure, 1.0 unit of insulin corresponds to 1/100^{th} of a milliliter. Said another way, each milliliter of insulin includes 100 units of insulin. Accordingly, 1.7 units of insulin is 1.7/100 of a milliliter or 0.017 milliliters. Therefore, a basal rate of 1.7 corresponds to 0.017 milliliters of insulin delivered per hour. If one wanted to subdivide this rate across the hour on a per-minute basis or on a minute package basis, it is easy to do so by simply taking the insulin dosage rates in column 2, multiplying this number by the number of minutes in each package (i.e., fifteen for a fifteen minute increment), and dividing by sixty. An experiment was carried out using the computer program model in accordance with the instant disclosure and using historical data described herein in order to output a series of suggested insulin dosage rates, which are summarized in FIG. 24.

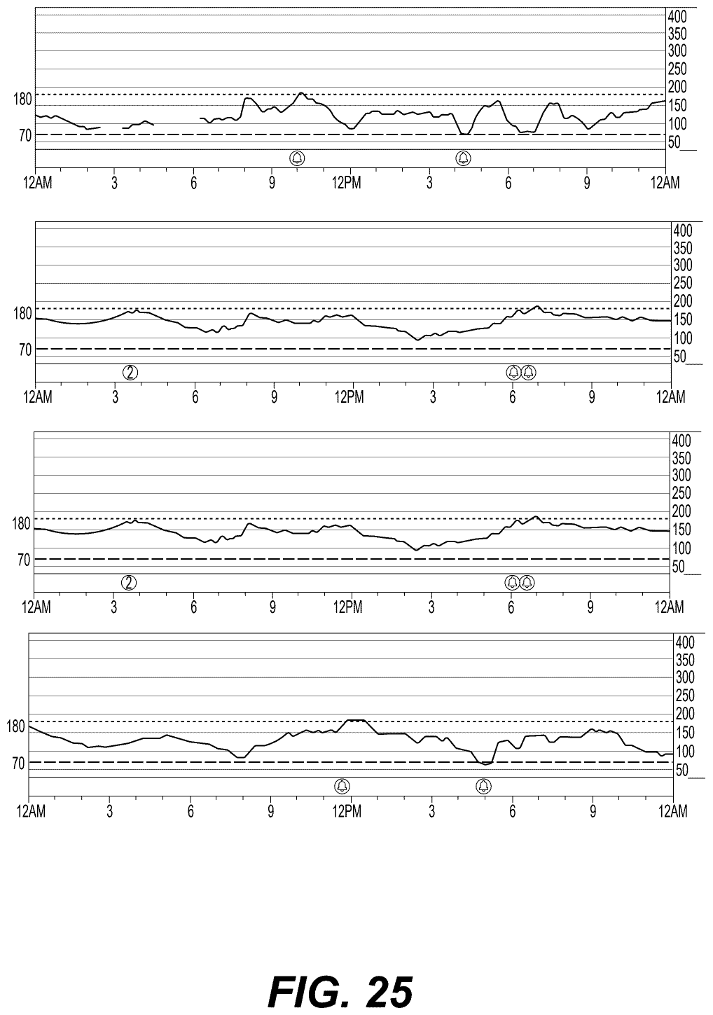

FIG. 25 depicts a chart tracking CGM data of a diabetic having programmed his diabetic pump to deliver insulin at the dosage rates depicted in FIG. 24. As is evident from the CGM data, recorded over four 24-hour periods, the diabetic's BGLs over each 24-hour period were relatively stabilized and fluctuated between 70 and 180 mg/dL (milligrams per deciliter).

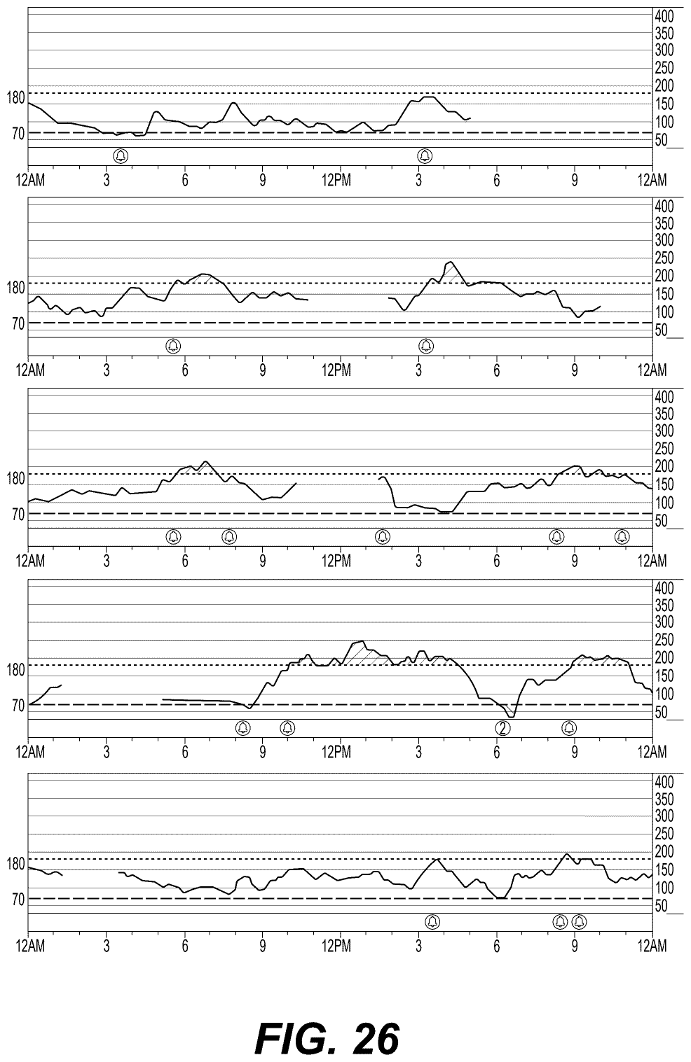

Turning to FIG. 26, not surprisingly, the more accurate the data is that is fed to the computer program model, the more accurate the suggested insulin basal dosage rates will be. As a result, it is important to ensure that the data fed to the computer program model is as accurate as is reasonably possible. This includes addressing missing data and incorrect data fed to the computer program model.

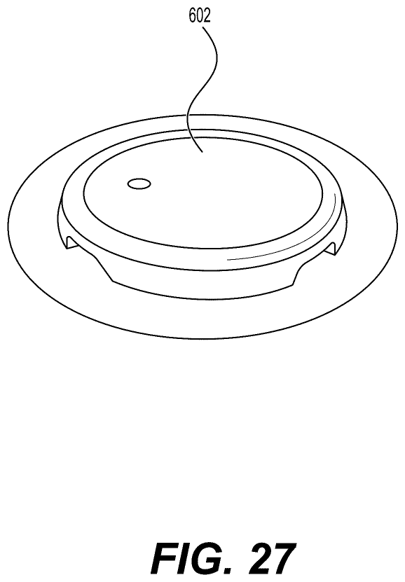

Referring to FIGS. 27-29, the latest CGM sensors from differing original equipment manufacturers (OEMs) are intended to be placed on the triceps of a person, but unfortunately, these sensors often fall off or get knocked off. CGM sensors that fall off or get knocked off create an expensive problem for OEMs that are forced to replace these CGM sensors for warranty claims after guaranteeing the CGM sensors will stay in place a full 10 days. Causes of the CGM sensors being displaced from a person's skin include, without limitation, adhesion degradation from sweat interposing the adhesive and skin, adhesion degradation from water permeation (such as from swimming or bathing), and adhesion degradation by succumbing to outside forces such as direct contact with the sensor. There are adhesives that a person can use, such as tape or a bandage, to help retain the CGM sensor in position. But it has been found that using these additional adhesives and/or bandages may result in blocking signals output from the CGM sensor throughout the day, which can lead to missing data during a 24-hour period, as shown graphically in FIG. 26. Accordingly, there is a need in the art to address problems associated with CGM sensor adhesion to a person's skin, while ensuring the CGM sensor is able to transmit its data in an uninterrupted manner to a receiver.

There are three main Continuous Glucose Sensors (CGS) in the Continuous Glucose Monitoring (CGM) space: (1) G6 and G7 sensors by Dexcom (see FIG. 27), (2) Libre 2 and Libre 3 sensors by Abbott, and (3) Guardian sensor by Medtronic. The latest sensors, Dexcom G6 and Libre 3 are round and smaller than previous sensors, but current regulatory approval requires these CGS devices to be applied only on the rear of a person's arm.

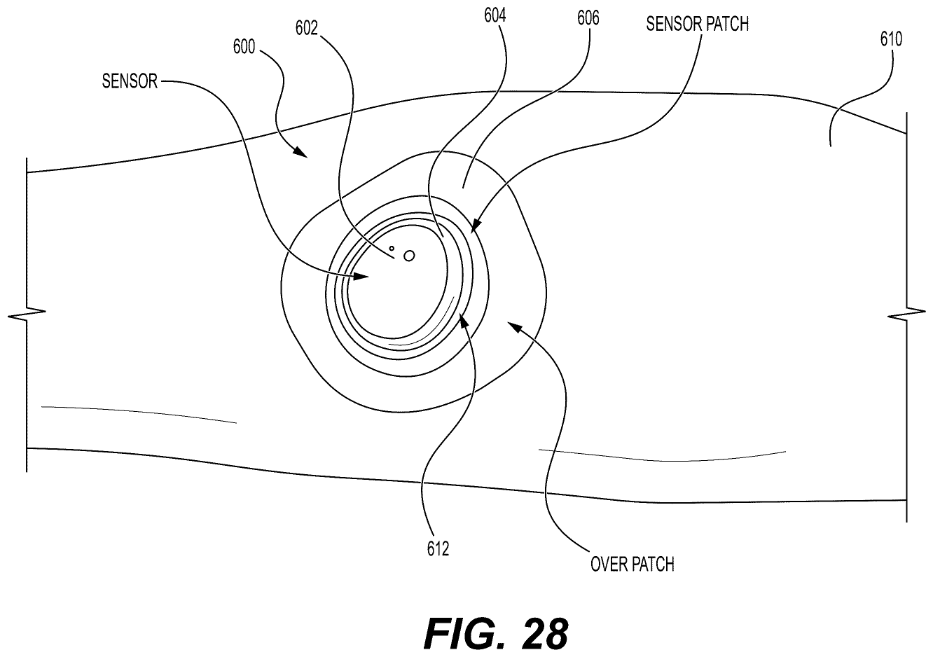

As depicted in FIG. 28, an exemplary CGM sensor 600 includes a primary housing 602 that protects internal circuitry (such as a transmitter and a glucose sensor) and a power source (not shown) such as a battery. By way of example, the sensor may comprise an interstitial fluid sensor configured to measure the concentration of glucose in said fluid. This primary housing 602 may be rigidly attached to a flexible sensor patch 604, the underside of which includes adhesive adapted to adhere the sensor patch to a person's skin 610 and thereby retain the position of the housing 602. It is known in the art to provide an over patch 606 that includes a central opening 612 to allow partial or total throughput of the primary housing 602, while providing a ring that circumscribes the primary housing. This ring is sized to concurrently overlap a portion of the sensor patch 604 and a portion of the person's skin 610 circumscribing the sensor patch. By way of example, the ring includes an adhesive on an underside thereof and is configured to concurrently bond to the top of the sensor patch 604 and the person's skin 610 in order to provide more skin contact area with an adhesive to bolster the temporary connection between the CGM sensor 600 and the person's skin. But the dimensions of the over patch 606 still allow the CGM sensor 600 to be contacted from its periphery, where contact with the periphery can hasten disengagement between the CGM sensor 600 and the person's skin.

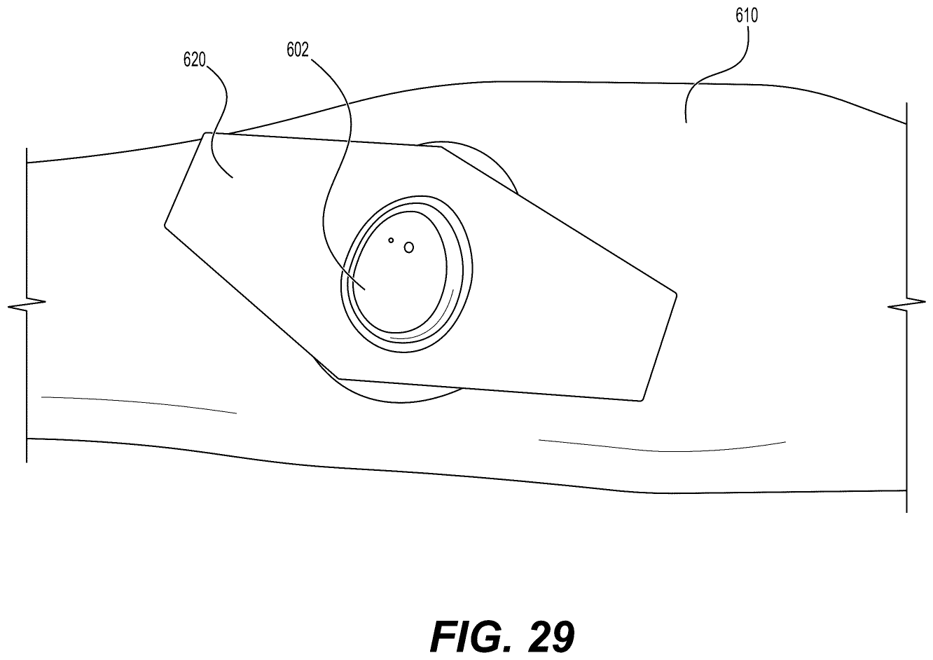

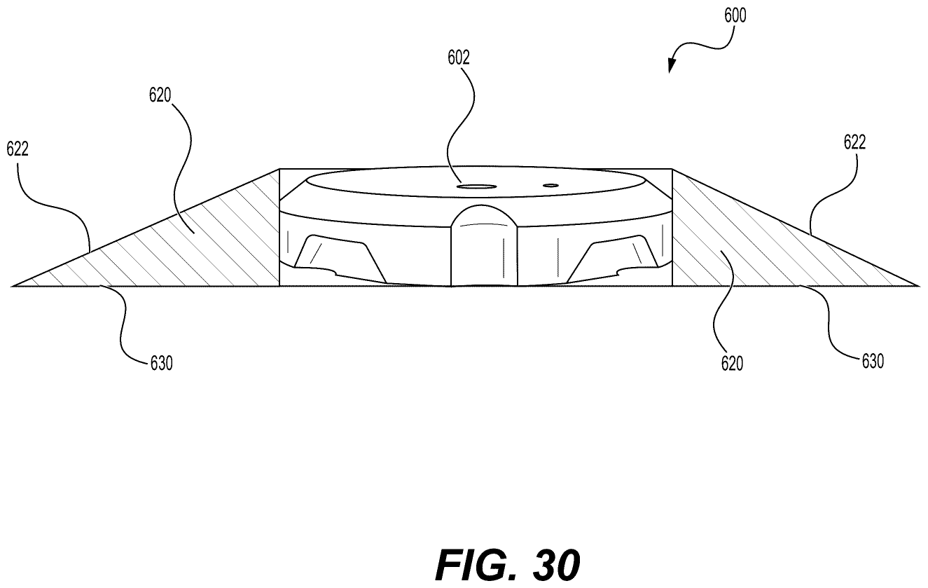

Referring to FIGS. 29 and 30, the instant disclosure includes a solution to the problem of maintaining a coupling between the CGM sensor 600 and a patient's skin 610 by providing a sensor guard 620. The exemplary sensor guard 620 may be a temporary guard that a person could wear for a certain amount of time and then can be removed from the person's skin when the CGM device 600 is removed or at another time prior to disengagement of the CGM device from the person's skin. For example, the sensor guard 620 could be worn while a person takes a shower, plays golf or any sport, or undertakes any physical activity that may lead to contact between objects and the CGM device 600. In exemplary form, the sensor guard 620 may be integrated as part of the primary housing 602 or the over patch 606, or may be a stand-alone item that is applied over and around the primary housing 602.

By way of example, the sensor guard 620 occupies a periphery of the primary housing 602 so that at most, only a top surface of the primary housing is readily accessible. In some applications, the sensor guard 620 may also cover the top of the primary housing 602 and/or include peripheral slats or openings that make one or more portions of the periphery of the primary housing accessible. In any event, the sensor guard 620 occupies a periphery of the primary housing and includes one or more exterior surfaces 622 that are angled, sloped, or tapered to provide a smooth transition between the person's skin 610 and at or near a top of the primary housing 602, or a top surface of the sensor guard. In exemplary form, this exterior surface may be manifest in a series of ramps peripherally distributed around the primary housing or may be manifest as part of a frustoconical shape transitioning between the skin and at or near the top of the primary housing. In this fashion, an attempt to contact a peripheral side of the primary housing 602, that might otherwise dislodge the primary housing from the skin 610, will be deflected via the sensor guard 620, so that any lateral force will be directed around the primary housing. Conversely, an attempt to contact the top of the primary housing 602 will be deflected by the sensor guard or, if the top surface of the primary housing is accessible, operate to push the primary housing against the skin 610. Accordingly, forces tending to displace the CGM device 600 from the skin 610 are reduced or mitigated altogether using a sensor guard 620.

In this exemplary configuration, the sensor guard 620 may comprise a frustoconical shape with an axially centered cylindrical cavity 626 (or other shaped cavity) to accommodate a primary housing 602 having a cylindrical shape. It should be noted that the internal cavity formed by the sensor guard 620 may embody any shape in order to accommodate primary housings 602 having differing shapes. A base 630 of the sensor guard may be planar or may have a deformable shape that allows the base to conform to the exterior surface shape of a patient's skin 610. More specifically, the base 630 may have applied thereto an adhesive configured to retain the sensor guard in place for a predetermined period of time, such time as not to exceed one month, for example. By way of further example, the base 630 may be bonded to an adhesive substrate that interposes the base and the adhesive coming into direct contact with the patient's skin 610.

While the depicted example of the sensor guard 620 embodies a frustoconical shape, it should be noted that any number of shapes are possible. For instance, the sensor guard 620 may comprises one or more ramps that are axially distributed about the periphery of the primary housing 602, with the inclined surface transitioning between the surface of the person's skin 610 and proximate a top of the sensor guard 620, primary housing 602, or intermediate structure.

In exemplary form, the sensor guard 620 may be fabricated from any number of materials including natural and synthetic fibers, whether or not woven into a fabric, plastics (including rubbers), composites, and ceramics. Likewise, the sensor guard 620 may comprises a uniform composition or may comprise a non-uniform composition where differing materials may be layered, staggered, alternated, oriented at differing positions. By way of example, the sensor guard may comprise a thermoplastic or a thermoset. Mover, the thermoset or thermoplastic may comprise an elastomer. The exemplary sensor guard 620 may be rigid, deformable, plastically deformable, elastically deformable, or otherwise.

The material(s) of the sensor guard 620 may be water impermeable, selectively water permeable, or water permeable. In this manner, the sensor guard 620 may include materials that swell when coming into contact with water or liquids in order to form a water-tight seal with the primary housing 602.

While the foregoing embodiment has been described as having an adhesive coupling the base 630 to a person's skin, it is also within the scope of the disclosure to omit the adhesive. Similarly, whether or not an adhesive is present, the sensor guard 620 may be mounted to a patient's body via application of a strap, a sleeve, a band, a belt-like device, or any other method for temporarily fixing the sensor guard 620 in position around a periphery of the CGM device 600.

It should be noted that the sensor guard 620 may be used with any bodily worn sensor and is not limited to application with only a CGM device 600.

Following from the above description, it should be apparent to those of ordinary skill in the art that, while the methods and apparatuses herein described constitute exemplary embodiments of the present invention, the invention described herein is not limited to any precise embodiment and that changes may be made to such embodiments without departing from the scope of the invention as defined by the claims. Additionally, it is to be understood that the invention is defined by the claims and it Is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

1. A method of creating an insulin dosing profile using at least one of a computer and a portable electronic device, the method comprising:
processing historical blood glucose level data output from a continuous glucose monitoring device over a first period of time for a first person to segment the historical blood glucose level data into basal controlled glucose patterns and bolus controlled glucose patterns;
using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump; and,
optionally, programming a controller of the insulin dosing pump in accordance with the suggested insulin dosing regimen.

2. The method of claim 1, wherein the first period of time includes at least seven consecutive days and preferably includes at least twenty consecutive days.

3. The method of claim 1, wherein:
processing historical blood glucose level data output from the continuous glucose monitoring device over the first period of time for the first person includes identifying missing blood glucose level data;
the method further comprises: interpolating, if necessary, to create blood glucose level data substituted for the missing blood glucose level data, thus creating a complete set of historical blood glucose level data for the first period of time;
segmenting the historical blood glucose level data includes segmenting the complete set of historical blood glucose level data into basal controlled glucose patterns and bolus controlled glucose patterns.

4. The method of claim 3, wherein identifying the missing blood glucose level data includes identifying whether the missing blood glucose level data is attributable to either a random error or a systematic error.

5. The method of claim 4, wherein the missing blood glucose level data is attributable to a systematic error, initiating a continuous glucose monitoring device calibration instruction.

6. The method of claim 4, wherein the missing blood glucose level data is attributable to a systematic error, initiating message to a user indicating the continuous glucose monitoring device is not properly working.

7. The method of claim 1, further comprising:
processing the historical blood glucose level data output from the continuous glucose monitoring device includes transforming raw data from the continuous glucose monitoring device into a structured dataset comprising the historical blood glucose level data.

8. The method of claim 7, wherein:
the historical blood glucose level data is comprised of data in a tabular form that includes glucose measurements and corresponding time stamps.

9. The method of claim 3, wherein:
interpolating, if necessary, to create blood glucose level data substituted for the missing blood glucose level data includes using linear interpolation to create the blood glucose level data where the missing data is attributable to a random occurrence.

10. The method of claim 3, further comprising:
applying a data smoothing operation to the complete set of historical blood glucose level data to mitigate large fluctuations in blood glucose level data at adjacent times.

11. The method of claim 10, wherein:
applying the data smoothing operation includes excluding blood glucose level data above a predetermined threshold.

12. The method of claim 1, wherein segmenting the historical blood glucose level data into basal controlled glucose patterns and bolus controlled glucose patterns includes identifying meal events using time stamps associated with the historical blood glucose level data.

13. The method of claim 12, wherein identifying meal events includes using time of day and rate of change of historical blood glucose level data.

14. The method of claim 12, wherein the bolus controlled glucose patterns comprises historical blood glucose level data attributable to meal events.

15. The method of claim 1, wherein segmenting the historical blood glucose level data into basal controlled glucose patterns and bolus controlled glucose patterns includes identifying a rate of change in the basal controlled glucose patterns.

16. The method of claim 15, wherein identifying the rate of change in the basal controlled glucose patterns includes segmenting the basal controlled glucose patterns into accelerated glucose level rate changes and decelerated glucose level rate change.

17. The method of claim 1, further comprising:
calculating a mean deviation for the historical blood glucose level data within the basal controlled glucose patterns; and,
establishing the suggested insulin dosing regimen for the insulin dosing pump using the mean deviation.

18. The method of claim 1, further comprising:
calculating a standard variation for the historical blood glucose level data within the basal controlled glucose patterns; and
establishing the suggested insulin dosing regimen for the insulin dosing pump using the standard deviation.

19. (canceled)

20. The method of claim 1, wherein segmenting the historical blood glucose level data into basal controlled glucose patterns and bolus controlled glucose patterns includes identifying a duration that historical blood glucose level data remained above a predetermined threshold, where values at or above the predetermined threshold results in the first person being hyperglycemic.

21. The method of claim 1, wherein segmenting the historical blood glucose level data into basal controlled glucose patterns and bolus controlled glucose patterns includes identifying a duration that historical blood glucose level data remained below a predetermined threshold, where values at or below the predetermined threshold results in the first person being hypoglycemic.

22. The method of claim 1, wherein establishing the suggested insulin dosing regimen for the insulin dosing pump includes using an input regarding a type of insulin dosed to the first person.

23. The method of claim 22, wherein the type of insulin dosed to the first person is at least one of very rapid-acting insulin, short acting insulin, intermediate acting insulin, and long acting insulin.

24. The method of claim 1, wherein establishing the suggested insulin dosing regimen for the insulin dosing pump includes using an input regarding an absorption rate of insulin dosed to the first person.

25. The method of claim 24, wherein the input regarding the absorption rate of insulin is derived from the historical blood glucose level data and the historical insulin dosing data.

26. The method of claim 1, wherein establishing the suggested insulin dosing regimen for the insulin dosing pump includes using an input regarding a physical condition of the first person.

27.-30. (canceled)

31. The method of claim 1, wherein establishing the suggested insulin dosing regimen for the insulin dosing pump includes using an input regarding a type of insulin pump the first person uses.

32. The method of claim 1, further comprising:
inputting the historical blood glucose level data output from the continuous glucose monitoring device over the first period of time for the first person; and,
inputting the historical insulin dosing for the first person over the first period of time.

33. The method of claim 1, further comprising:
processing additional historical blood glucose level data output from the continuous glucose monitoring device over a second period of time for the first person to segment the additional historical blood glucose level data into additional basal controlled glucose patterns and additional bolus controlled glucose patterns;
using the historical blood glucose level data within the basal controlled glucose patterns, the additional historical blood glucose level data within the additional basal controlled glucose patterns, the historical insulin dosing data over the first period of time, and historical insulin dosing data over the second period of time, but excluding using the historical blood glucose level data within the bolus controlled glucose patterns and the additional historical blood glucose level data within the additional bolus controlled glucose patterns, to establish a suggested revised insulin dosing regimen for the insulin dosing pump; and,
optionally, reprogramming the insulin dosing pump in accordance with the suggested revised insulin dosing regimen.

34.-35. (canceled)

36. A method for executing a computer application, the method comprising:
running a computer application on a portable electronic device comprising executing a software application embodied on the portable electronic device which causes the portable electronic device to perform the steps of:
processing historical blood glucose level data output from a continuous glucose monitoring device over a first period of time for a first person to segment the historical blood glucose level data into basal controlled glucose patterns and bolus controlled glucose patterns;
using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump; and,
optionally, programming a controller of the insulin dosing pump in accordance with the suggested insulin dosing regimen.

37.-70. (canceled)

71. A computer program product for establishing a glucose dosing regimen for an insulin pump, the computer program product comprising:
a non-transitory computer readable medium encoded with computer executable code, the code configured to enable the execution of:
processing historical blood glucose level data output from a continuous glucose monitoring device over a first period of time for a first person to segment the historical blood glucose level data into basal controlled glucose patterns and bolus controlled glucose patterns; and,
using the historical blood glucose level data within the basal controlled glucose patterns and historical insulin dosing data over the first period of time, but excluding using the historical blood glucose level data within the bolus controlled glucose patterns, to establish a suggested insulin dosing regimen for an insulin dosing pump.

72.-105. (canceled)

106. An insulin pump with a controller programmed with executable code configured to enable execution of the following acts:
processing historical blood glucose level data output from a continuous glucose monitoring device over a first period of time for a first person to segment the historical blood glucose level data into basal controlled glucose patterns and bolus controlled glucose patterns;
using the historical blood glucose level data within the basal controlled glucose patterns and historical insulin dosing data over the first period of time, but excluding using the historical blood glucose level data within the bolus controlled glucose patterns, to establish an insulin dosing regimen; and,
dosing insulin consistent with the insulin dosing regimen.

107.-157. (canceled)
